(19) United States
(12) Patent Application Publication
(10) Pub. No.: US 2024/0261258 A1
(43) Pub. Date: Aug. 8, 2024

(54) Presurgical Perineural Administration of Resiniferatoxin For Reduction of Post-Operative Pain

(71) Applicant: Sorrento Therapeutics, Inc., San Diego, CA (US)

(72) Inventors: Alexis Nahama, San Diego, CA (US); Alvaro Cisternas, San Diego, CA (US)

(73) Assignee: Sorrento Therapeutics, Inc., San Diego, CA (US)

(21) Appl. No.: 18/561,529

(22) PCT Filed: May 17, 2022
(86) PCT No.: PCT/US2022/029584

§ 371 Date: Nov. 16, 2023

Related U.S. Application Data
(60) Provisional application No. 63/189,947, filed on May 18, 2021.

(51) Int. Cl. A61K 31/357; A61K 31/445; A61P 29/00; A61P 41/00

(52) U.S. Cl. CPC A61K 31/357; A61K 31/445; A61P 29/00; A61P 41/00

(57) ABSTRACT

Disclosed herein are methods, and compositions for use in such methods, of presurgical administering of resiniferatoxin (RTX) perineurally to provide peri-surgical and post-surgical benefits, including but not limited to decreasing post-surgical pain.

### CROSS-REFERENCE TO RELATED APPLICATIONS

This application claims priority to U.S. Provisional Application No. 63/189,947, filed May 18, 2021, the disclosure of which is hereby incorporated by reference in its entirety.

### FIELD OF THE INVENTION

The present disclosure provides methods for improving post-surgical recovery processes comprising administering resiniferatoxin (RTX) perineurally, and resiniferatoxin for use in such methods.

### I. INTRODUCTION AND SUMMARY

RTX acts as an ultrapotent analog of capsaicin, the pungent principal ingredient of the red pepper. RTX is a tricyclic diterpene isolated from certain species of Eurphorbia. A homovanillyl group is an important structural feature of capsaicin and is the most prominent feature distinguishing resiniferatoxin from typical phorbol-related compounds. Native RTX has the following structure:

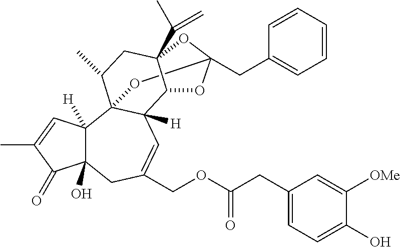

RTX and analog compounds such as tinyatoxin and other compounds, (20-homovanillyl esters of diterpenes such as 12-deoxyphorbol 13-phenylacetate 20-homovanillate and mezerein 20-homovanillate) are described in U.S. Pat. Nos. 4,939,194; 5,021,450; and 5,232,684. Other resiniferatoxin-type phorboid vanilloids have also been identified (Szallasi et al. (1999) Brit. J Pharmacol. 128:428-434).

RTX is known as a TrpV1 agonist. TrpV1, the transient receptor potential cation channel subfamily V member 1 (also known as Vanilloid receptor-1 (VR1)) is a multimeric cation channel prominently expressed in nociceptive primary afferent neurons (Caterina et al. (1997) Nature 389:816-824; Tominaga et al. (1998) Neuron 21:531-543). Activation of TrpV1 typically occurs at the nerve endings via application of painful heat and is up regulated during certain types of inflammatory stimuli. Activation of TrpV1 in peripheral tissues by a chemical agonist results in the opening of calcium channels and the transduction of a pain sensation (Szallasi et al. (1999) Mol. Pharmacol. 56:581-587). However, direct application of certain TrpV1 agonists to the cell body of a neuron (ganglion) expressing TrpV1 opens calcium channels and triggers a cascade of events leading to programmed cell death (“apoptosis”) (Karai et al. (2004) J. of Clin. Invest. 113:1344-1352).

Surgery induced trauma or insult to a body causes cytokines to be released and inflammation processes to be activated. Recovery from surgery is generally painful, and certain orthopedic surgeries and amputations are known to be especially painful, frequently requiring extended pain management physical therapy post-surgery. While pain management is an extensive and refined area of medical care, treatment for severe and chronic pain often involves opiates and other narcotics that carry the risk of a variety of undesirable side effects including addiction.

Accordingly, there is a need in the art to develop methods and compositions to reduce pain associated with surgery and/or to improve the surgical subject's recovery post-surgery. Provided herein are methods of administering RTX perineurally, to a subject in need of surgery, for providing peri-surgical and post-surgical benefits.

Accordingly, the following exemplary embodiments are provided.

Embodiment 1 is a method of preparing a subject for surgery, comprising perineurally administering RTX to a subject in need of surgery.

Embodiment 2 is Resiniferatoxin (RTX) for use in a method of preparing a subject for surgery, the method comprising perineurally administering RTX to a subject in need of surgery.

Embodiment 3 is the method or RTX for use of embodiment 1 or 2, wherein the method provides a peri-surgical or a post-surgical benefit to the subject.

Embodiment 4 is the method or RTX for use of any one of the preceding embodiments, wherein the method reduces post-surgical pain in the subject.

Embodiment 5 is the method or RTX for use of any one of the preceding embodiments, wherein the method accelerates post-surgical recovery time of the subject.

Embodiment 6 is the method or RTX for use of any one of the preceding embodiments, wherein the method reduces the amount of peri-surgical anesthetic administered to the subject.

Embodiment 7 is the method or RTX for use of any one of the preceding embodiments, wherein the method reduces the amount of post-surgical anesthetic administered to the subject.

Embodiment 8 is the method or RTX for use of any one of the preceding embodiments, wherein the method reduces inflammation caused by trauma at a surgical site.

Embodiment 9 is the method or RTX for use of any one of the preceding embodiments, wherein the method reduces cytokine release caused by trauma at a surgical site.

Embodiment 10 is the method or RTX for use of any one of the preceding embodiments, wherein nerve signaling pathways are ablated in the subject.

Embodiment 11 is the method or RTX for use of any one of the preceding embodiments, wherein the subject is being prepared for a surgical procedure.

Embodiment 12 is the method or RTX for use of any one of the preceding embodiments, wherein the subject is placed under anesthesia prior to the administration of RTX.

Embodiment 13 is the method or RTX for use of any one of the preceding embodiments, wherein the surgery occurs within 1, 2, 3, 4, 5, or 6 hours of administering RTX.

Embodiment 14 is the method or RTX for use of any one of the preceding embodiments, wherein the subject is in need of an orthopedic surgery or an amputation.

Embodiment 15 is the method or RTX for use of any one of the preceding embodiments, wherein the subject is in need of surgical intervention on a bone in the back, hand, arm or leg.

Embodiment 16 is the method or RTX for use of any one of the preceding embodiments, wherein the RTX is administered as an infiltration.

Embodiment 17 is the method or RTX for use of any one of the preceding embodiments, wherein the RTX is administered to a single site.

Embodiment 18 is the method or RTX for use of any one of the preceding embodiments, wherein the RTX is administered to a plurality of sites.

Embodiment 19 is the method or RTX for use of any one of the preceding embodiments, wherein the RTX is administered locally to a surgical site.

Embodiment 20 is the method or RTX for use of any one of the preceding embodiments, wherein RTX is administered as a nerve block.

Embodiment 21 is the method or RTX for use of any one of the preceding embodiments, wherein RTX is administered as a nerve block at the sciatic nerve and/or the femoral nerve.

Embodiment 22 is the method or RTX for use of any one of the preceding embodiments, wherein the subject is a mammal.

Embodiment 23 is the method or RTX for use of any one of the preceding embodiments, wherein the subject is a cat, dog, horse, pig, ruminant, cow, sheep, goat, or domesticated mammal.

Embodiment 24 is the method or RTX for use of any one of the preceding embodiments, wherein the subject is a human.

Embodiment 25 is the method or RTX for use of any one of the preceding embodiments, wherein the method comprises administering a dose of 0.1 μg to 100 μg of RTX, or a dose of 2 μg to 50 μg of RTX.

Embodiment 26 is the method or RTX for use of embodiment 25 wherein the dose of RTX ranges from 0.1-1 μg, 1-2 μg, 2-5 μg, 5-10 μg, 10-20 μg, 20-30 μg, 30-40 μg, 40-50 μg, 50-60 μg, 60-70 μg, 70-80 μg, 80-90 μg, or 90-100 μg.

Embodiment 27 is the method or RTX for use of any one of the preceding embodiments, wherein the method comprises administering a pharmaceutical formulation comprising the RTX and a pharmaceutically acceptable carrier.

Embodiment 28 is the method or RTX for use of embodiment 27, wherein the pharmaceutically acceptable carrier comprises water.

Embodiment 29 is the method or RTX for use of embodiment 27 or 28, wherein the pharmaceutically acceptable carrier comprises polysorbate 80.

Embodiment 30 is the method or RTX for use of any one of embodiments 27-29, wherein the pharmaceutically acceptable carrier comprises polyethylene glycol.

Embodiment 31 is the method or RTX for use of any one of embodiments 27-30, wherein the pharmaceutically acceptable carrier comprises a sugar or sugar alcohol.

Embodiment 32 is the method or RTX for use of embodiment 27-31, wherein the pharmaceutically acceptable carrier comprises mannitol.

Embodiment 33 is the method or RTX for use of embodiment 27-32, wherein the pharmaceutically acceptable carrier comprises dextrose.

Embodiment 34 is the method or RTX for use of any one of embodiments 27-33, wherein the pharmaceutically acceptable carrier comprises a pharmaceutically acceptable buffer.

Embodiment 35 is the method or RTX for use of embodiment 27-34, wherein the pharmaceutically acceptable carrier comprises a phosphate buffer.

Embodiment 36 is the method or RTX for use of any one of embodiments 27-35, wherein the pharmaceutical formulation has a pH in the range of 6 to 7.6.

Embodiment 37 is the method or RTX for use of embodiment 36, wherein the pharmaceutical formulation has a pH in the range of 6 to 6.4, 6.3 to 6.7, 6.4 to 6.8, 6.8 to 7.2, 7 to 7.4, or 7.2 to 7.6.

Embodiment 38 is the method or RTX for use of embodiment 36, wherein the pharmaceutical formulation has a pH of 6.5 or 7.2.

Embodiment 39 is the method or RTX for use of any one of embodiments 27-38, wherein the pharmaceutically acceptable carrier comprises a pharmaceutically acceptable salt.

Embodiment 40 is the method or RTX for use of embodiment 39, wherein the pharmaceutically acceptable salt is NaCl.

Embodiment 41 is the method or RTX for use of any one of embodiments 27-40, wherein the concentration of RTX in the pharmaceutical formulation is in the range of 0.02 to 300 μg/ml.

Embodiment 42 is the method or RTX for use of embodiment 41, wherein the concentration of RTX in the pharmaceutical formulation is in the range of 0.02-0.1 μg/ml, 0.1-1 μg/ml, 1-5 μg/ml, 5-10 μg/ml, 10-20 μg/ml, 20-50 μg/ml, 50-100 μg/ml, 100-150 μg/ml, 150-200 μg/ml, 200-250 μg/ml, or 250-300 μg/ml.

Embodiment 43 is the method or RTX for use of embodiment 41, wherein the concentration of RTX in the pharmaceutical formulation is in the range of 150 to 250 μg/ml, or is about 200 μg/ml.

Embodiment 44 is the method or RTX for use of embodiment 41, wherein the concentration of RTX in the pharmaceutical formulation is in the range of 0.1-200 μg/ml, optionally wherein the concentration of RTX in the pharmaceutical formulation is in the range of 0.1-50 μg/ml.

Embodiment 45 is the method or RTX for use of any one of the preceding embodiments, wherein the RTX is administered in an injection volume of 0.05-10 ml, optionally wherein the injection volume is in the range of 0.05-0.2 ml, 0.2-0.5 ml, 0.5-1 ml, 1-2 ml, 2-5 ml, or 5-10 ml.

Embodiment 46 is the method or RTX for use of any one of the preceding embodiments, wherein the method further comprises administering a local anesthetic.

Embodiment 47 is the method or RTX for use of embodiment 46, wherein the local anesthetic is an amino-amide anesthetic.

Embodiment 48 is the method or RTX for use of embodiment 46, wherein the local anesthetic is bupivacaine.

Embodiment 49 is the method or RTX for use of any one of the preceding embodiments, wherein the method is a method of reducing post-operative pain.

Embodiment 50 is the method or RTX for use any one of the preceding embodiments, wherein the method is a method of accelerating post-surgical recovery time of the subject.

### II. BRIEF DESCRIPTION OF THE DRAWINGS

FIGS. 1A-J show density of CGRP immunopositive axons in representative confocal images of ipsilateral (left; A-D) and contralateral (right; E-H) sciatic nerve from dogs post-TPLO and after different treatment and doses of RTX. Each bar represents the mean±SEM. Data were analyzed by one-way ANOVA followed by Tukey post hoc test; *p<0.05. FIGS. 1I-J are bar graphs of the density of CGRP immunopositive axons, (density of nerve fibers in mm/mm3) in ipsilateral (I) and contralateral (J) sciatic nerves as measured in n subjects in each group.

FIGS. 2A-J show density of SP nerve axons in representative confocal images of ipsilateral (left; A-D) and contralateral (right; E-H) sciatic nerve from dogs post-TPLO and after different treatment and doses of RTX. Each bar represents the mean±SEM. Data were analyzed by one-way ANOVA followed by Tukey post hoc test; *p<0.05. FIGS. 2I-J are bar graphs of the density of SP nerve axons (density of nerve fibers in mm/mm3) in ipsilateral (I), and contralateral (J) sciatic nerves as measured in n subjects in each group.

FIGS. 3A-J show CGRP immunopositive axon density in representative confocal images of ipsilateral (left; A-D) and contralateral (right; E-H) femoral nerve from dogs post-TPLO surgery and after different treatment and doses of RTX. Each bar represents the mean±SEM. Data were analyzed by one-way ANOVA followed by Tukey post hoc test; *p<0.05. FIGS. 3I-J are bar graphs of the density of CGRP immunopositive axons (density of nerve fibers in mm/mm3) in ipsilateral (I), and contralateral (J) femoral nerves as measured in n subjects in each group.

FIGS. 4A-J show the density of SP-immunoreactive axons in representative confocal images showing the presence of CGRP axons from ipsilateral (A-D) and contralateral (E-H) canine femoral nerves from dogs post-TPLO and after treatment of RTX. Each bar represents the mean±SEM. FIGS. 4I-J are bar graphs of the density of SP nerve axons (density of nerve fibers in mm/mm3) in ipsilateral (I), and contralateral (J) femoral nerves as measured in n subjects in each group.

### III. DETAILED DESCRIPTION

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents, which may be included within the invention as defined by the appended claims.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition. In some embodiments, “about” encompasses variation within 10%, 5%, 2%, 1%, or 0.5% of a stated value.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The section headings used herein are for organizational purposes only and are not to be construed as limiting the desired subject matter in any way. In the event that any literature incorporated by reference contradicts any term defined in this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### A. Definitions

“Perineural administration” as used herein is administration to a nerve fiber between the nerve ending and the cell body. For example, perineural administration encompasses injection of an agent in sufficient proximity to a nerve fiber between the nerve endings and the nerve cell bodies that the agent contacts the nerve fiber.

As used herein, “ablation” or “neural ablation” refers to the destruction or inactivation of a part of a biological tissue (e.g., nerve), e.g., using an agent such as RTX. To be clear, ablation of a nerve encompasses partial destruction or inactivation.

A “ruminant” is a mammal that has a rumen. Examples of ruminants include, but are not limited to cattle, sheep, antelopes, deer, and giraffes.

The terms “or a combination thereof” and “or combinations thereof” as used herein refers to any and all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

“Or” is used in the inclusive sense, i.e., equivalent to “and/or,” unless the context requires otherwise.

### B. Exemplary Methods and Compositions for Use

The present disclosure is based in part on the realization that pre-surgical perineural administration of RTX can provide a reduction in the post-surgery pain experienced by subjects. The reduction can affect either or both of the intensity and duration of the pain. In some embodiments, pre-surgical perineural administration of RTX may accelerate the post-surgical recovery time of the subject. In some embodiments, a reduction of post-surgical pain is relative to the extent of pain (e.g., average or median subjectively reported pain) that results from a version of the procedure without administration of RTX and consistent with the applicable standard of care. In some embodiments, an acceleration of post-surgical recovery time is relative to the recovery time (e.g., average or median recovery time) that would result from a version of the procedure without administration of RTX and consistent with the applicable standard of care.

Without wishing to be bound by any particular theory, RTX may modulate the heat or inflammation component of the pain signal (not necessarily acute pain). While preserving pressure and motor coordination, without necessarily affecting acute pain, treatment with RTX at the time of surgery can reduce post surgical pain by (i) limiting the development of and the discomfort associated with, post-surgical tissue inflammation; (ii) blocking further development of neurogenic inflammation (e.g., for days or weeks); and/or preventing nerve growth such as the development or proliferation of additional nerve endings in healing tissue, which can lead to surgical incision or neuroma pain. In some embodiments, pre-surgical perineural administration may reduce inflammatory nerve signals. In some embodiments, pre-surgical perineural administration may reduce signaling by heat-sensitive neurons. In some embodiments, pre-surgical perineural administration may reduce nerve growth (e.g., development or proliferation of additional nerve endings) in healing tissue after surgery. In some embodiments, pre-surgical perineural administration may reduce surgical incision or neuroma pain after surgery.

Without wishing to be bound by any particular theory, perineurally administering RTX may ablate nerves or nerve endings and interrupt pain signaling pathways. In some embodiments, the pre-surgical interventions disclosed herein may interrupt cytokine release that results from tissue trauma at the site of surgery. In some embodiments, the pre-surgical interventions disclosed herein may reduce inflammation that results from tissue trauma at the site of surgery. In some embodiments, the methods may reduce or prevent the occurrence of neuropathic pain from nerve trauma during surgeries.

The interruption in pain signaling can provide any one or more of a variety of peri-surgical and post-surgical benefits. In some embodiments, less pain experienced by the subject allows for a reduction in the amount of peri-surgical anesthetic administered to the subject. In some embodiments, the amount of post-surgical anesthetic administered to the subject is reduced. In some embodiments, pre-surgical perineural administration of RTX reduces the need for pain medications, such as opioids, during surgery, or after-surgery, or both.

In some embodiments, post-surgery, the subject experiences less pain, less inflammation and less swelling, allowing generally for more comfortable convalescence.

### C. Subjects

The compositions and methods described herein are for use with any subject in whom RTX is effective, e.g., able to bind and activate TrpV1 or a homolog thereof, and who is in need of surgery. In some embodiments, the subject is a mammal. In some embodiments, the mammal is a human. In some embodiments, the mammal is a domesticated mammal. In some embodiments, the mammal is a cat. In some embodiments, the mammal is a dog. In some embodiments, the mammal is a ruminant. In some embodiments, the mammal is a horse, cow, pig, sheep, or goat.

In some embodiments, the subject may be in need of orthopedic surgery. In some embodiments, the subject is in need of surgical intervention on a bone in the back, hand, arm or leg. In some embodiments, the subject may require an amputation of a finger, hand, forearm, arm, toe, foot, leg, or portion thereof (e.g., for the leg, above or below the knee). Other surgeries known to be particularly painful include, but are not limited to fracture repair, open heart surgery, gallbladder removal, liposuction, bone marrow donation, dental implants, joint replacements (such as knee, hip, shoulder, or elbow), abdominal hysterectomy, spinal fusions and spinal reconstructions, myomectomy, or proctocolectomy.

In some embodiments, the subject may be in need of pain management methods that reduce or eliminate the need for potentially addictive opioids or other narcotic drugs.

### D. Sites and Timing of Administration

RTX may be administered before surgery, while a subject is being prepared for a surgical procedure. In some embodiments, RTX is administered 6 hours, or 5 hours or 4 hours, or 3 hours, or 2 hours, or 1 hour, or 30 min before surgery. In some embodiments, the patient is placed under anesthesia before administration of RTX.

RTX may be administered perineurally to one or more than one site, depending on the site of the surgery. In some embodiments, the RTX is administered perineurally to a single site. In some embodiments, the RTX is administered locally to a surgical site.

In some embodiments, the RTX is administered perineurally to the femoral nerve. In some embodiments, the RTX is administered perineurally to the sciatic nerve. In some embodiments, the RTX is administered perineurally to the saphenous nerve. In some embodiments, the RTX is administered perineurally to the radial nerve. In some embodiments, the RTX is administered perineurally to the ulnar nerve. In some embodiments, the RTX is administered perineurally to the median nerve. In some embodiments, the RTX is administered perineurally to the musculocutaneous nerve. In some embodiments, the RTX is administered perineurally to the palmar digital nerve. The sciatic nerve runs from the lower back to the legs (hind legs in the case of quadrupeds). The saphenous nerve is the largest cutaneous branch of the femoral nerve and is located in the lower leg (lower hindlimb in the case of quadrupeds). The femoral nerve is located in the upper thigh of the leg (upper hindlimb in the case of quadrupeds). The radial nerve is located in the upper arm (upper forelimb in the case of quadrupeds). The ulnar nerve is located in the forearm and the hand (forelimb in the case of quadrupeds). The median nerve is located in the upper arm (forelimb in the case of quadrupeds). The musculocutaneous nerve is located in the arm (forelimb in the case of quadrupeds) and branches off from the median nerve in the middle of the humerus. The palmar digital nerves are located in the hand (in the terminal segment of the forelimb in the case of quadrupeds).

In some embodiments, the RTX is administered perineurally to a plurality of sites. For example, treating the sciatic and saphenous nerves would block leg pain, treating the ulnar and palmar digital nerves would block hand pain, and treating the radial and median nerves would block upper arm pain. In some embodiments, the RTX is administered perineurally to a plurality of sites that collectively correspond to sensory input from one or more digits. In some embodiments, the RTX is administered perineurally to a plurality of sites that collectively correspond to sensory input from a foot or hand. In some embodiments, the RTX is administered perineurally to a plurality of sites that collectively correspond to sensory input from a forelimb (e.g., forearm or lower leg). In some embodiments, the RTX is administered perineurally to a plurality of sites that collectively correspond to sensory input from a limb (e.g., arm or leg). In some embodiments, the RTX is administered perineurally to a plurality of sites that collectively correspond to sensory input from a joint. In some embodiments, a plurality of sites includes a plurality of branches of a nerve (e.g., the dorsal and palmar branches of the ulnar nerve).

In some embodiments, the (RTX) is administered by injection. Injections may be performed, e.g., using a 1 cc syringe, or more generally, a size of syringe appropriate for the dosage volume. In some embodiments, the RTX is administered as an infiltration.

In some embodiments, the method further comprises administering a local anesthetic (i.e., in addition to the RTX). The local anesthetic may be administered to the same nerve as the RTX. The local anesthetic may be administered separately or in the same composition as the RTX. In some embodiments, the local anesthetic is an amino-amide anesthetic, such as lidocaine, mepivacaine, prilocaine, bupivacaine, etidocaine, ropivacaine, or levobupivacaine. In some embodiments, the local anesthetic is bupivacaine.

### E. Dosage

In some embodiments, the RTX is administered at a dose of 0.1-100 μg. In some embodiments, the dose of RTX ranges from 0.1-0.5 μg, 0.5-1 μg, 1-2 μg, 2-5 μg, 5-10 μg, 10-20 μg, 20-30 μg, 30-40 μg, 40-50 μg, 50-60 μg, 60-70 μg, 70-80 μg, 80-90 μg, or 90-100 μg. For example, e.g., in de-clawed cats, a total dose of 2.5 μg may be perineurally administered to one or both forelimbs. In humans, in some embodiments, a dose of up to 25 μg (e.g., 5-10 μg, 10-15 μg, 15-20 μg, or 20-25 μg; or about 5, 10, 15, 20, or 25 μg) is administered. In some embodiments, the RTX is administered (e.g., to a human) at a dose of 2-50 μg. In some embodiments, a 2-, 3-, or 4-point nerve block technique is used, with a total dosage in any of the ranges listed above, such as a total dosage of 0.5-1 μg, 1-2 μg, 2-5 μg, 5-10 μg, 10-15 μg, 15-20 μg, or 20-25 μg.

The dosage and volume can be adjusted depending on the proximity of the site of administration to the nerve fiber. For example, where ultrasound or a nerve stimulator is used to ensure that the site of administration is very close to the nerve, a lower dose and volume can be used. Alternatively, a nerve block such as a scapular or sciatic block can be accomplished using a larger volume such as 3-5 ml to ensure contact with the desired nerves. Notably, RTX is specific for the TRPV1 receptor and therefore does not affect non-target nerves such as motor neurons that do not have enough TRPV1 receptors to be sensitive to RTX.

### F. Formulations

Multiple examples of formulations of RTX are available in the literature. See, e.g., Ueda et al. (2008) J. of Cardiovasc. Pharmacol. 51:513-520, and US 2015/0190509 A1. Any suitable formulation of RTX for parenteral administration (e.g., injection) may be used.

In some embodiments, the RTX, which may be at the dosages discussed above, is administered with a pharmaceutically acceptable carrier. In some embodiments, the pharmaceutically acceptable carrier comprises water. In some embodiments, the pharmaceutically acceptable carrier comprises polysorbate 80. In some embodiments, the pharmaceutically acceptable carrier comprises polyethylene glycol. In some embodiments, the pharmaceutically acceptable carrier comprises sugar or sugar alcohol. In some embodiments, the pharmaceutically acceptable carrier comprises mannitol. In some embodiments, the pharmaceutically acceptable carrier comprises dextrose. In some embodiments, the pharmaceutically acceptable carrier comprises a pharmaceutically acceptable buffer. In some embodiments, the pharmaceutically acceptable carrier comprises a phosphate buffer. In some embodiments, the pharmaceutically acceptable carrier comprises a pharmaceutically acceptable salt. In some embodiments, the pharmaceutically acceptable carrier comprises NaCl. In some embodiments, the pharmaceutically acceptable carrier comprises an organic solvent such as ethanol or DMSO, e.g., as a minority or residual component used as an aid in dissolving RTX before dilution in a primarily aqueous composition.

The concentration of RTX in the formulation may be any suitable value for delivery of the intended dose. In some embodiments, the concentration of RTX in the pharmaceutical formulation is in the range of 0.1 to 300 μg/ml. In some embodiments, the concentration of RTX in the pharmaceutical formulation is in the range of 0.1-1 μg/ml, 1-5 μg/ml, 5-10 μg/ml, 10-20 μg/ml, 10-30 μg/ml, 20-30 μg/ml, 20-50 μg/ml, 50-100 μg/ml, 100-150 μg/ml, 150-200 μg/ml, 200-250 μg/ml, or 250-300 μg/ml. In some embodiments, the concentration of RTX in the pharmaceutical formulation is in the range of 150 to 250 μg/ml, or 200 μg/ml. In some embodiments, the concentration of RTX in the pharmaceutical formulation is in the range of 0.1 to 200 μg/ml, such as 0.1 to 50 μg/ml or 50 to 200 μg/ml, or about 0.1, 0.2, 0.5, 1, 1.5, 2, 5, 10, 20, 25, 50, 100, or 200 μg/ml.

The formulation may have any pH suitable for perineural administration. In some embodiments, the pharmaceutical formulation comprising the RTX and a pharmaceutically acceptable carrier has a pH in the range of 6 to 7.6. In some embodiments, the pharmaceutical formulation comprising the RTX and a pharmaceutically acceptable carrier has a pH in the range of 6 to 6.4, 6.3 to 6.7, 6.4 to 6.8, 6.8 to 7.2, 7 to 7.4, or 7.2 to 7.6. In some embodiments, the pharmaceutical formulation comprising the RTX and a pharmaceutically acceptable carrier has a pH of 6.5 or 7.2.

In some embodiments, the formulation comprises polysorbate 80 and dextrose. In some embodiments, the concentration of polysorbate 80 is 0.03-7% w/v. In some embodiments, the concentration of polysorbate 80 is 2-4% w/v, and/or the concentration of dextrose is 4-6% w/v. In some embodiments, the concentration of polysorbate 80 is 3% w/v, and/or the concentration of dextrose is 5% w/v. In some embodiments, in any of the foregoing formulations, the concentration of RTX may be 10-30 μg/ml, such as 10 μg/ml or 25 μg/ml. The formulation may further comprise a buffer, such as phosphate buffer (e.g., sodium phosphate buffer). In some embodiments, the concentration of phosphate buffer is 10-50 mM. In some embodiments, the concentration of phosphate buffer is 10-30 mM. In some embodiments, the concentration of phosphate buffer is 10 mM. In some embodiments, the concentration of phosphate buffer is 30 mM. The formulation may have a pH in the range of 7-7.5, such as about 7.2. In some embodiments, in any of the foregoing formulations, the concentration of RTX may be 10-30 mcg/ml, such as 10 mcg/ml or 25 mcg/ml. In some embodiments, the formulation further comprises phosphate buffer, e.g., at a concentration and pH shown for phosphate buffer in Table 1. In some embodiments, the formulation further comprises NaCl, e.g., at a concentration shown for NaCl in Table 1. When both are present, the phosphate buffer and NaCl may be (but are not necessarily) present at a combination of concentrations and phosphate buffer pH shown for an individual formulation.

Exemplary formulations of RTX are shown in the following table.

TABLE 1
Exemplary RTX Solution Formulations

| Formu- | | | |
|---|---|---|---|
| lation | | Component | |
| Number | Formulation Components | Concentration | |
| 1 | RTX | 200 | mcg/mL |
| | Polysorbate 80 | 7.0% | w/v |
| | Dextrose | 0.8% | w/v |
| | 30 mM Phosphate Buffer w/0.44% NaCl | 30 mM, pH 7.2 | |
| 2 | RTX | 200 | mcg/mL |
| | Polyethylene Glycol 300 | 3.0% | v/v |
| | Polysorbate 80 | 0.1% | w/v |
| | Dextrose | 0.8% | w/v |
| | 10 mM Phosphate Buffer w/0.73% NaCl | 10 mM, pH 6.5 | |
| 3 | RTX | 200 | mcg/mL |
| | Polyethylene Glycol 300 | 30.0% | v/v |
| | Polysorbate 80 | 1.0% | w/v |
| | 10 mM Phosphate Buffer w/0.86% NaCl | 10 mM, pH 6.5 | |
| 4 | RTX | 200 | mcg/mL |
| | Polyethylene Glycol 300 | 30.0% | v/v |
| | Polysorbate 80 | 0.04% | w/v |
| | 10 mM Phosphate Buffer w/0.88% NaCl | 10 mM, pH 6.5 | |
| 5 | RTX | 200 | mcg/mL |
| | Polysorbate 80 | 3.0% | w/v |
| | Dextrose | 0.8% | w/v |
| | 30 mM Phosphate Buffer w/0.54% NaCl | 30 mM, pH 7.2 | |
| 6 | RTX | 200 | mcg/mL |
| | Polysorbate 80 | 3.0% | w/v |
| | Mannitol | 0.8% | w/v |
| | 30 mM Phosphate Buffer w/0.54% NaCl | 30 mM, pH 7.2 | |
| 7 | RTX | 200 | mcg/mL |
| | Polysorbate 80 | 7.0% | w/v |
| | Mannitol | 0.8% | w/v |
| | 30 mM Phosphate Buffer w/0.45% NaCl | 30 mM, pH 7.2 | |
| 8 | RTX | 200 | mcg/mL |
| | Polyethylene Glycol 300 | 3.0% | v/v |
| | Polysorbate 80 | 0.1% | w/v |
| | Mannitol | 0.8% | w/v |
| | 10 mM Phosphate Buffer w/0.74% NaCl | 10 mM, pH 6.5 | |
| 9 | RTX | 200 | mcg/mL |
| | Polyethylene Glycol 300 | 3.0% | v/v |
| | Polysorbate 80 | 0.1% | w/v |
| | Dextrose | 3.0% | w/v |
| | 10 mM Phosphate Buffer w/0.34% NaCl | 10 mM, pH 6.5 | |
| 10 | RTX | 200 | mcg/mL |
| | Polyethylene Glycol 300 | 3.0% | v/v |
| | Polysorbate 80 | 0.1% | w/v |
| | Mannitol | 3.0% | w/v |
| | 10 mM Phosphate Buffer w/0.36% NaCl | 10 mM, pH 6.5 | |
| 11 | RTX | 200 | mcg/mL |
| | Polysorbate 80 | 0.03% | w/v |
| | Dextrose | 0.05% | w/v |
| | 30 mM Phosphate Buffer w/0.54% NaCl | 30 mM, pH 7.2 | |
| 12 | RTX | 200 | mcg/mL |
| | Polysorbate 80 | 3.0% | w/v |
| | Dextrose | 5.0% | w/v |
| | 30 mM Phosphate Buffer w/0.54% NaCl | 30 mM, pH 7.2 | |
| 13 | RTX | 25 | mcg/mL |
| | Polysorbate 80 | 3.0% | w/v |
| | Dextrose | 5.0% | w/v |
| | 30 mM Phosphate Buffer w/0.54% NaCl | 30 mM, pH 7.2 | |
| 14 | RTX | 25 | mcg/mL |
| | Polysorbate 80 | 0.03% | w/v |
| | Dextrose | 0.05% | w/v |
| | 30 mM Phosphate Buffer w/0.54% NaCl | 30 mM, pH 7.2 | |
| 15 | RTX | 100 | mcg/mL |
| | Polysorbate 80 | 0.03% | w/v |
| | Dextrose | 0.05% | w/v |
| | 30 mM Phosphate Buffer w/0.54% NaCl | 30 mM, pH 7.2 | |
| 16 | RTX | 200 | mcg/mL |
| | Polysorbate 80 | 7.0% | w/v |
| | Dextrose | 5.0% | w/v |

| | |
|---|---|
| 30 mM Phosphate Buffer w/0.54% NaCl | 30 mM, pH 7.2 |

In some embodiments, formulations in Table 1 include dextrose. In embodiments, the concentration of dextrose is 0.05-5% w/v. In some embodiments, the concentration of dextrose is 0.8-5 w/v. In some embodiments, the concentration of dextrose is 0.05% w/v. In some embodiments, the concentration of dextrose is 0.8% w/v. In some embodiments, the concentration of dextrose is 3.0% w/v. In some embodiments, the concentration of dextrose is 5.0% w/v.

In some embodiments, formulations in Table 1 include mannitol. In some embodiments, the concentration of mannitol is 0.8-3.0% w/v. In some embodiments, the concentration of mannitol is 0.8% w/v. In some embodiments, the concentration of mannitol is 3.0% w/v.

In some embodiments, the dextrose or mannitol is omitted from a formulation shown in Table 1.

In some embodiments, the concentration of RTX in a formulation shown in Table 1 is adjusted to any of the RTX concentrations or concentration ranges disclosed herein. For example, in some embodiments, the concentration of RTX in a formulation shown in Table 1 is adjusted to 0.3-200 mcg/ml. In some embodiments, the concentration of RTX in a formulation shown in Table 1 is 200 mcg/ml. In some embodiments, the concentration of RTX in a formulation shown in Table 1 is 0.3-100 mcg/ml. In some embodiments, the concentration of RTX in a formulation shown in Table 1 is 100 mcg/ml. In some embodiments, the concentration of RTX in a formulation shown in Table 1 is adjusted to 0.3-50 mcg/ml. In some embodiments, the concentration of RTX in a formulation shown in Table 1 is 25 mcg/ml. As another example, in some embodiments, the concentration of RTX in a formulation shown in Table 1 is adjusted to 0.3-15 mcg/ml. As another example, in some embodiments, the concentration of RTX in a formulation shown in Table 1 is adjusted to 0.5-10 mcg/ml. As another example, in some embodiments, the concentration of RTX in a formulation shown in Table 1 is adjusted to 0.6-1.5 mcg/ml. The dextrose or mannitol is omitted from any such formulation having an adjusted RTX concentration.

In some embodiments, the concentration of RTX in a formulation shown in Table 1 is adjusted to any of the RTX concentrations or concentration ranges disclosed herein. For example, in some embodiments, the concentration of RTX in a formulation shown in Table 1 is adjusted to 10-50 μg/ml. As another example, in some embodiments, the concentration of RTX in a formulation shown in Table 1 is adjusted to 10-30 μg/ml. As another example, in some embodiments, the concentration of RTX in a formulation shown in Table 1 is adjusted to 20-30 μg/ml. As another example, in some embodiments, the concentration of RTX in a formulation shown in Table 1 is adjusted to 25 μg/ml. In some embodiments, the concentration of RTX in a formulation shown in Table 1 is adjusted to a concentration in the range of 0.1 to 100 μg/ml, or 0.1 to 50 μg/ml, such as 0.1 to 25 μg/ml, 25 to 50 μg/ml, or 50 to 100 μg/ml, or about 0.1, 0.2, 0.5, 1, 1.5, 2, 5, 10, 20, 25, 50, or 100 μg/ml.

The formulations in Table 1 may be prepared according to the following exemplary methods, which are provided for formulations 3 and 5 but may be adapted to the other formulations by one skilled in the art. Formulation 3 may be made by adding 46 mg sodium phosphate monobasic monohydrate, 94.7 mg sodium phosphate dibasic anhydrous, and 860 mg NaCl to a 100 ml volumetric flask. 50 ml of water for injection (WFI) is added to dissolve the components in the flask, followed by addition of 1.0 g of polysorbate 80, to form the aqueous component. 20 mg of RTX is added to the aqueous component in the volumetric flask, and pH is adjusted with hydrochloric acid/sodium hydroxide to 7.2. Then 30 mL of PEG 300 is added and the solution is sonicated to dissolve the solids. It should be noted that RTX will sometimes precipitate at the interface of aqueous solution and PEG initially, but will go back into solution upon sonication. The full mixture in the flask is diluted to volume (100.00 ml) with water (WFI) and this is mixed by an inversion process. The full formulation is filtered through a 0.2 μm polytetrafluoroethylene (PTFE) filter.

Formulation 5 may be made by adding 138 mg sodium phosphate monobasic monohydrate, 284.1 mg sodium phosphate dibasic anhydrous, and 540 mg NaCl to a 100 ml volumetric flask. 50 ml of water for injection (WFI) is added to dissolve the components in the flask, followed by addition of 3.0 g of polysorbate 80, and 800 mg of dextrose to form the aqueous component. 20 mg of RTX is added the aqueous component in the volumetric flask, and pH is adjusted with hydrochloric acid/sodium hydroxide to 7.2. The solution is then sonicated to dissolve all the solids. (Alternatively, the RTX may be initially dissolved in a small volume of ethanol or DMSO, and this solution may then be added to the aqueous component.) The full mixture in the flask is diluted to volume (100.00 ml) with water (WFI) and this is mixed by an inversion process. The full formulation is filtered through a 0.2 μm PTFE filter.

A formulation according to Formulation 11 is prepared using 200 mcg RTX, 300 mcg Polysorbate 80 (using commercially-available polysorbate 80); 5.4 mg of sodium chloride, 500 mcg of dextrose, 1.38 mg sodium phosphate monobasic monohydrate, 2.84 mg sodium phosphate dibasic anhydrous, and water (WFI) to 1 mL, then pH is adjusted with hydrochloric acid/sodium hydroxide to 7.2. As noted above, the dextrose may be omitted.

A formulation according to Formulation 13 is prepared using 25 mcg RTX, 30 mg Polysorbate 80 (using commercially-available polysorbate 80); 5.4 mg of sodium chloride, 50 mg of dextrose, 1.38 mg sodium phosphate monobasic monohydrate, 2.84 mg sodium phosphate dibasic anhydrous, water (WFI) to 1 mL, then pH is adjusted with hydrochloric acid/sodium hydroxide to 7.2. As noted above, the dextrose may be omitted.

In some embodiments, the pharmaceutical formulation is in a unit dosage form. In such form, the preparation is subdivided into unit doses containing appropriate quantities of the active component. The unit dosage form can be a packaged preparation, the package containing discrete quantities of formulation, such as in vials, ampoules, or pre-loaded syringes. Also, the unit dosage form can be, e.g., a solution or a lyophilized composition for reconstitution.

Further details on techniques for formulation and administration may be found in Gennaro, A., Ed., Remington's Pharmaceutical Sciences, 18th Ed. (1990) (Mack Publishing Co., Easton, Pa.).

### IV. EXAMPLES

### A. Improved Inflammation and Pain after a Tibial Plateau Osteotomy (TPLO) in a Dog

A TPLO (Tibial Plateau Osteotomy) was performed on a single dog. Before surgery, 25 μg RTX was administered perineurally to the femoral and sciatic nerves. The procedure included cutting the bone in the leg, changing the angle of the knee articulation, screwing in a plate, and resewing muscle that was cut to access the knee.

Surgery on the dog was successful and smoother than expected, with no reaction from the dog during cutting of the bone and no need to deepen the general anesthesia as is usually done at that stage of the surgery. In addition, there was no need to increase the opioid dose for the last part of the surgery. No additional pain medications was given when the dog woke up.

After waking up, all of the dog's motor functions were preserved. In addition, the pain at manipulation, inflammation, and swelling as determined using a standardized scale used by veterinarians for post-surgical pain assessment, were each lower than expected. Within a couple of hours, the dog was up and walking. The dog was able to put the affected leg down from time to time when ambulating but, as would be expected, the leg was not fully weight-bearing.

### B. Tolerability and Safety of RTX Administered as a Nerve Block in the Hindlimb and Local Application in Healthy Dogs Undergoing TPLO Surgery

This example provides safety data related to the tolerability of RTX administered as a nerve block pre-surgically and as a local application on the surgical site intraoperatively in healthy dogs undergoing tibial plateau levelling osteotomy (TPLO). Pharmacological behavior of RTX after a perineural injection and local application in the hindlimb and the long-term effects on bone healing was also observed.

Fourteen adult female beagle dogs, weighing 11.9±0.6 kg and between 3-5 years of age, were enrolled in this study. Each animal was randomly assigned to one of four groups. Four dogs (group 1) received a femoral and sciatic nerve block with 12.5 μg RTX per nerve (25 μg RTX total) diluted with sterile saline for a total volume of 2 mL (half volume per block). Another four dogs (group 2) received a femoral and a sciatic nerve block with 3.12 μg RTX per nerve block (6.25 μg RTX total) diluted with sterile saline for a total volume of 2 mL (half volume per block). Group 3 (4 animals) received a femoral and sciatic nerve block with 5 mg bupivacaine total (standard of care) plus a total of 50 μg RTX (total volume of 4 mL, concentration 12.5 μg/mL) applied locally to the cut on the tibia for 10 min. Group 4 (2 animals) was the control group and received a femoral and a sciatic nerve block with 5 mg bupivacaine total (standard of care).

TABLE 2
Animal assignment and treatment.

| | | | | | | Bupiva- |
|---|---|---|---|---|---|---|
| Dog | | Body | RTX | | | caine |
| ID | Sex | weight | total dose | | ROA | total dose |
| 0271 | Female | 11.4 kg | 25 | μg | F/Sc nerve block | 0 mg |
| 3837 | Female | 11.8 kg | 25 | μg | F/Sc nerve block | 0 mg |
| 5635 | Female | 12.6 kg | 25 | μg | F/Sc nerve block | 0 mg |
| 2968 | Female | 12.4 kg | 25 | μg | F/Sc nerve block | 0 mg |
| 4681 | Female | 11.4 kg | 6.25 | μg | F/Sc nerve block | 0 mg |
| 5686 | Female | 11.6 kg | 6.25 | μg | F/Sc nerve block | 0 mg |
| 2561 | Female | 11.8 kg | 6.25 | μg | F/Sc nerve block | 0 mg |
| 3743 | Female | 12.4 kg | 6.25 | μg | F/Sc nerve block | 0 mg |
| 9501 | Female | 12.2 kg | Up to 50 | μg | Local | 5 mg |
| 3007 | Female | 11.2 kg | Up to 50 | μg | Local | 5 mg |
| 2272 | Female | 11.8 kg | Up to 50 | μg | Local | 5 mg |
| 3722 | Female | 11.2 kg | Up to 50 | μg | Local | 5 mg |
| 5100 | Female | 12.6 kg | 0 | μg | N/A | 5 mg |
| 7812 | Female | 12.8 kg | 0 | μg | N/A | 5 mg |

### Pharmacokinetics

Blood (4-5 mL) was collected at the beginning of the study for complete blood count and chemistry parameters. Another 2 mL sample was collected at time 0 (baseline), 5, 15, 45, 60, and 90 minutes, and at 2, 3, 4, 6, and 8 hours after RTX administration for pharmacokinetic analysis to determine how plasma levels of RTX change over time. Dog plasma samples were extracted using an organic solvent mixture, dried under nitrogen, reconstituted and analyzed by reversed-phase HPLC. The mobile phase was nebulized using heated nitrogen in a Z-spray source/interface set to electrospray positive ionization mode. The ionized compounds are detected using tandem mass spectrometry or MS/MS.

### Radiological Evaluation

Radiographs of the stifle were performed before surgery, the day after surgery, at day 28, and at day 56. Dogs were sedated to allow for correct positioning of the leg and to minimize stress from handling. Standardized TPLO views (lateral and caudal-cranial) were acquired. An MRI and CT were also performed on the operated leg after the animals were euthanized and the surgical plate was removed (day 56).

### Sedation and Anesthesia

Dogs were sedated and anesthetized using standard protocols and following the American College of Veterinary Analgesia and Anesthesia guidelines. A physical exam was performed prior to surgery and a complete blood count and chemistry profile were submitted. Food was withheld for 8-10 hours before surgery and offered again 4-6 hours after recovery from anesthesia.

Diphenhydramine (2.2 mg/kg) was given IM after induction. The peripheral IV catheter was used for administration of fluid (Lactate Ringer's solution (LRS) at 5 mL/kg/h), and intraoperative drugs when necessary (hydromorphone and/or ketamine and/or dexmedetomidine for analgesia, atropine in case of bradycardia, dopamine or dobutamine in case of hypotension). Cefazolin 22 mg/kg was administered intravenously prior to surgery and every 60-90 minutes during surgery.

Femoral and sciatic nerve blocks with RTX or bupivacaine were performed after induction of general anesthesia, and followed by surgery (TPLO). RTX was administered at least 15 minutes prior to any cut made to the bone. Dogs in group 3 received RTX during surgery via direct application on the bone.

At the end of surgery, animals were allowed to recover. Following extubation, animals were supervised until able to stand and walk steadily, then monitored for the first 4 hours following the procedure. Food and water were given at 4-6 hours if recovery was smooth and uneventful. All subjects received oral carprofen 2.2 mg/kg twice a day for 10 days and trazodone 5-7 mg/kg twice a day, until the end of the study.

### RTX Administration

Perineural injections (femoral and sciatic nerve block) of RTX were performed before surgery under general anesthesia and application of RTX over the bone incision was done during the surgical procedure. The nerve blocks were applied at after administration of anesthesia and least 15 minutes before surgery.

For the perineural injections, the animal was placed in right lateral recumbency and the lateral and medial aspects of proximal area of the left hindlimb were clipped and prepared using an aseptic technique. A caudal approach was used for the sciatic nerve block. The ultrasound transducer was placed over the lateral aspect of the biceps femoris, just distal to the greater trochanter and oriented craniocaudally. After obtaining a short axis view of the sciatic nerve, a sheathed needle for electrolocation was inserted caudally in-plane with the ultrasound transducer until the tip of the needle reached the nerve. The correct location of the needle was confirmed by a positive response (twitches of the leg) after activation of the nerve locator using a current of 0.3-0.5 mA. The entire volume of RTX (1 mL) was then administered over 90 seconds followed by 0.4 mL of saline to clear the injection line of the sheathed needle.

The femoral nerve was identified by placing the ultrasound transducer transversally on the medial surface of the thigh midway along the femur. After identification or the femoral artery, the transducer was moved proximally until the superficial circumflex iliac artery was visualized. The sheathed needle was then inserted in a cranial to caudal direction using an in-plane technique until the tip of the needle was near the femoral nerve. The correct location of the needle was confirmed by a positive response (twitches of the leg) after activation of the nerve locator using a current of 0.3-0.5 mA. The entire volume of RTX (1 mL) was then administered over 90 seconds followed by 0.4 mL of saline to clear the injection line of the sheathed needle.

Animals in group 3 received RTX locally. After the surgical dissection of the tibia was completed and the TPLO plate was secured in place, 4 mL (50 μg) of RTX were placed on a small area of a sterile 4×4 gauze. The area of the gauze loaded with RTX was then locally applied over the TPLO plate on the bone incision site for 10 minutes.

### Surgical Procedure

The proximal tibia of the left hindlimb was approached medially. A craniomedial arthrotomy was performed and the joint capsule was closed. The popliteal muscle was elevated from the caudal aspect of the proximal tibia. A TPLO saw was used to osteotomize the proximal tibia and rotate its plateau. A TPLO plate was secured to the medial side of the tibia with screws. The surgical wound was lavaged with warm, sterile saline and the deep fascia, subcutaneous layer, and skin was closed. The internal layers of the skin were closed with absorbable suture material and the skin using a simple interrupted pattern with non-absorbable suture material. A protective bandage was applied to the surgical site and an E-collar placed until the skin sutures were removed.

### Post-Surgical Evaluations

Before the subjects underwent surgery, baseline data including a physical examination, hindlimb motor evaluation, hindlimb sensory evaluation, site of injection evaluation, and surgical site evaluation were collected. During the 8-week post-operative follow-up period on day 1, 2, 3, 14, 28, and 56 all the above parameters were reevaluated by a researcher who was blinded to the treatment.

After observing the animal in its cage and after evaluating the subject walking, the overall pain was scored using a visual analog scale (VAS) and the short form of the Glasgow composite pain scale (GCPS). The VAS consisted of a straight line 10 cm long with the left end, 0 cm, indicating no pain and the right end, 10 cm, indicating unbearable pain. The user placed a mark on the line that best represented the intensity of the subject's pain. Animals with a VAS greater than 4 cm and a GCPS greater that 6 were considered painful and received intramuscular hydromorphone at 0.1 mg/kg.

The injection site of RTX or local anesthetic was evaluated by visual examination (normal, red, swollen) and palpation (no pain, mild, severe). The surgical site was evaluated by visual examination (normal, red, swollen) and with the aid of an algometer. With the dog in lateral recumbency, the tip of the algometer was placed on the surgical site and pressure was manually applied until the animal moved the leg. During the baseline data collection, several measurements were taken until 3 measurements were within ±1 Newton (N). The number recorded corresponded to the average of these 3 values. After surgery, the force was applied only once. Data were collected from both the left (treated) and the right (non-treated) hindlimbs.

The withdrawal reflex and proprioception were assessed and scored as normal, decreased, or absent. The motor function was evaluated by encouraging the dog to stand and walk. Weight bearing standing and walking was scored as: normal, decreased (dog uses the leg but prefers the non-operated leg), or absent (dog avoids using the operated leg).

### Data Analysis

Individual data are presented for each animal. Due to the small number of dogs per group, only descriptive statistics for each group are described.

### RTX Plasma Concentrations (Pg/mL)

RTX Plasma concentrations (pg/mL) were measured in the dogs following administration of RTX solution as femoral and sciatic nerve blocks.

Results are presented in Table 3 (high dose group), Table 4 (low dose group), Table 5 (local group), and Table 6 (placebo group). Plasma levels were below the quantifiable limit at all timepoints for all dogs that received the local application (Table 5) and for those in the control (placebo) group (Table 6). Regardless of dose, plasma levels of RTX did not reach detectable levels until at least 90 minutes after administration, except for one dog in group 1 (subject 5635). For the low dose group, RTX plasma levels fell to below detectable after 3-4 hours, whereas for the high dose group levels were still detectable at 8 hours.

TABLE 3
Plasma concentrations (pg/mL) for RTX in Group 1
(High Dose) dogs.

| Total Dose | Volume | | Dog ID | | | |
|---|---|---|---|---|---|---|
| (μg) | (mL) | Time | 0271 | 2968 | 3837 | 5635 |
| 25 | 1 | Predose | BQL | BQL | BQL | BQL |
| | | 0 min | BQL | BQL | BQL | 397 |
| | | 5 min | BQL | BQL | BQL | 100 |
| | | 15 min | BQL | BQL | BQL | 96.4 |
| | | 45 min | BQL | BQL | BQL | BQL |
| | | 60 min | BQL | BQL | BQL | BQL |
| | | 90 min | BQL | 55.1 | BQL | 81.4 |
| | | 2 h | 115 | 1,790 | 95.6 | 1,740 |
| | | 3 h | 198 | 239 | 131 | 290 |
| | | 4 h | 154 | 150 | 395 | 221 |
| | | 6 h | 105 | 94.1 | 182 | 130 |
| | | 8 h | 65.6 | 62.3 | 117 | 99.1 |

BQL—Below the Quantifiable Limit < 50.0 pg/mL

TABLE 4
Plasma concentrations (pg/mL) for RTX in Group 2
(Low Dose) dogs.

| Total Dose | Volume | | Dog ID | | | |
|---|---|---|---|---|---|---|
| (μg) | (mL) | Time | 2561 | 3743 | 4681 | 5686 |
| 6.25 | 1 | Predose | BQL | BQL | BQL | BQL |
| | | 0 min | BQL | BQL | BQL | BQL |
| | | 5 min | BQL | BQL | BQL | BQL |
| | | 15 min | BQL | BQL | BQL | BQL |
| | | 45 min | BQL | BQL | BQL | BQL |
| | | 60 min | BQL | BQL | BQL | BQL |
| | | 90 min | 85.0 | BQL | BQL | BQL |
| | | 2 h | 60.7 | BQL | BQL | BQL |
| | | 3 h | BQL | 84.7 | BQL | 50.0 |
| | | 4 h | BQL | BQL | BQL | 50.6 |
| | | 6 h | BQL | BQL | BQL | BQL |
| | | 8 h | BQL | BQL | BQL | BQL |

BQL—Below the Quantifiable Limit < 50.0 pg/mL

TABLE 5
Plasma concentrations (pg/mL) for RTX in Group 3
(Local) dogs.

| Dose | Volume | | Dog ID | | | |
|---|---|---|---|---|---|---|
| (μg) | (mL) | Time | 2272 | 3007 | 3722 | 9501 |
| 50 | 4 | Predose | BQL | BQL | BQL | BQL |
| | | 0 min | BQL | BQL | BQL | BQL |
| | | 5 min | BQL | BQL | BQL | BQL |
| | | 15 min | BQL | BQL | BQL | BQL |
| | | 45 min | BQL | BQL | BQL | BQL |
| | | 60 min | BQL | BQL | BQL | BQL |
| | | 90 min | BQL | BQL | BQL | BQL |
| | | 2 h | BQL | BQL | BQL | BQL |
| | | 3 h | BQL | BQL | BQL | BQL |
| | | 4 h | BQL | BQL | BQL | BQL |
| | | 6 h | BQL | BQL | BQL | BQL |
| | | 8 h | BQL | BQL | BQL | BQL |

BQL—Below the Quantifiable Limit < 50.0 pg/mL

TABLE 6
Plasma concentrations (pg/mL) for
RTX in Group 4 (Placebo) dogs.

| | | |
|---|---|---|
| Dose | Volume | Dog ID |

| (μg) | (mL) | Time | 5100 | 7812 |
|---|---|---|---|---|
| 0 | 1 | Predose | BQL | BQL |

| | | | |
|---|---|---|---|
| 0 | min | BQL | BQL |
| 5 | min | BQL | BQL |
| 15 | min | BQL | BQL |
| 45 | min | BQL | BQL |
| 60 | min | BQL | BQL |
| 90 | min | BQL | BQL |
| 2 | h | BQL | BQL |
| 3 | h | BQL | BQL |
| 4 | h | BQL | BQL |
| 6 | h | BQL | BQL |
| 8 | h | BQL | BQL |

BQL—Below the Quantifiable Limit < 50.0 pg/mL

### Clinical Observations During RTX Instillation Under General Anesthesia

General anesthesia was successfully induced in all dogs without complications. The end tidal isoflurane ranged between 1-1.8%, 1-1.7%, 0.9-1.8%, and 0.9-1.3% for group 1, 2, 3, and 4, respectively. Intraoperative propofol was administered IV due to a light plane of anesthesia to all four dogs in group 1 (range 1-4 mg/kg), two in group 2 (2-2.5 mg/kg), and two in group 3 (0.6-2 mg/kg). Intraoperative hydromorphone was administered at 0.5 mg/kg IV during closure of the subcutaneous layer to two dogs in group 1, two in group 2, and one in group 3. None of the dogs in group 1, and only one dog in group 2, required dobutamine to treat hypotension (common adverse effect of inhalant anesthetics) after RTX administration. This intervention was necessary for two dogs in group 3 and both dogs in group 4. A mild to moderate increase in heart rate and blood pressure was observed approximately 15 to 25 minutes after RTX administration in group 1 and 2 subjects. Subject 2968 (group 1) did not have any increased in the cardiovascular parameters at the time listed above; however, an increase in heart rate from 90 bpm to 168 bpm with a mean blood pressure increase from 60 mmHg to 172 mmHg occurred 100 minutes after RTX administration when the dog's leg was stretched during aseptic preparation for surgery (plasma concentration was 1790 μg/mL at 2 hour timepoint). The subject received appropriate intervention and recovered without further complications. It was also noticed that this dog was in estrus at the time of surgery.

Immediately after extubation, one dog in group 2 and one in group 3 received 1 μg/kg dexmedetomidine IV to treat dysphoria. One dog in group 2 received naloxone IV titrated to effect (total of 0.008 mg/kg) due to prolonged recovery. After extubation, dogs in group 1 and 2 presented polypnea which persisted until the body temperature decreased to 94-95° F. When a heating source was applied to increase, the body temperature, all dogs moved away from the heated area. In three subjects of group 1 (0271, 2968, and 5635) the hypothermia persisted for over 24 hours, without any clinical signs. All three animals acted normally, eating and drinking regularly, without pain upon palpation of the surgical site, and no abnormalities in their bloodwork, which was repeated at day 2 for subject 2968 and day 3 for subject 2968. The body temperature normalized without any intervention.

### Post-Surgical Pain Evaluations

Within 2 hours of surgery, when the dogs resumed walking, two dogs in group 1 used the operated leg normally and the other two placed the leg down (toe touching). A similar toe touching behavior was observed in three dogs from group 2, while the other dog did not use the treated leg. None of the dogs in groups 3 and 4 used the operated leg immediately after surgery.

Weight bearing was absent or decreased while standing and walking in all dogs (including controls) for 1-3 days after surgery. All dogs had normal scores at day 14, 28, and 56 except for one in group 2.

In group 1, two dogs started bearing weight while standing at day 2 and the other two dogs at day 3.

In group 2, three dogs started bearing weight at day 3 and the fourth dog at day 2; however, the latter dog did not bear weight standing and walking at day 1. One dog showed decreased weight bearing walking and standing at day 14 for unrelated causes: an interdigital cyst was found and treated with topical antibiotic.

In group 3, one dog had normal weight bearing standing at day 1 and decreased while walking, which normalized at day 2. The others had a decreased score standing and walking for 2-3 days.

In group 4, one dog had decreased weight bearing standing and walking for 3 days and the other dog had a decreased score standing at day 1, decreased weight bearing standing at day 1 and absent/decreased while walking at day 1, 2, and 3 (Table 7).

The withdrawal reflex was normal in all dogs at all time points. Proprioception was decreased at day 1 in three out of four dogs in group 1; in two out of three dogs in group 2; in two out of four dogs in group 3; and in one out of two dogs in group 4. Proprioception was not assessed in one dog from group 2 on day 1 because the dog was not weight bearing and would not tolerate extension of the leg (Table 7).

TABLE 7
Weight bearing, withdrawal reflex, and proprioception assessment
the day of surgery and at day 1, 2, and 3 after surgery.

| Parameter observed | | Group | | | |
|---|---|---|---|---|---|
| or scored | | 1. High (25 μg) | 2. Low (6.25 μg) | 3. Local (50 μg) | 4. Placebo |
| Use of leg within | | Normal: 2/4 | Intermittent toe | Absent: 4/4 | Absent: 2/2 |
| 2 hours of surgery | | Intermittent toe | touching: 3/4; | | |
| | | touching: 2/4 | Absent: 1/4 | | |
| Weight bearing | Day 1 | Normal: 0/4 | Normal: 0/4 | Normal: 1/4 | Normal: 0/2 |
| while standing | | Decreased: 4/4 | Decreased: 3/4 | Decreased: 3/4 | Decreased: 2/2 |
| | | Absent: 0/4 | Absent: 1/4 | Absent: 0/4 | Absent: 0/2 |
| | Day 2 | Normal: 2/4 | Normal: 2/4 | Normal: 2/4 | Normal: 1/2 |
| | | Decreased: 2/4 | Decreased: 2/4 | Decreased: 2/4 | Decreased: 1/2 |
| | | Absent: 0/4 | Absent: 0/4 | Absent: 0/4 | Absent: 0/2 |
| | Day 3 | Normal: 4/4 | Normal: 4/4 | Normal: 4/4 | Normal: 1/2 |
| | | Decreased: 0/4 | Decreased: 0/4 | Decreased: 0/4 | Decreased: 1/2 |
| | | Absent: 0/4 | Absent: 0/4 | Absent: 0/4 | Absent: 0/2 |
| Weight bearing | Day 1 | Normal: 0/4 | Normal: 0/4 | Normal: 0/4 | Normal: 0/2 |
| while walking | | Decreased: 3/4 | Decreased: 3/4 | Decreased: 4/4 | Decreased: 1/2 |
| | | Absent: 1/4 | Absent: 1/4 | Absent: 0/4 | Absent: 1/2 |
| | Day 2 | Normal: 0/4 | Normal: 2/4 | Normal: 1/4 | Normal: 0/2 |
| | | Decreased: 4/4 | Decreased: 2/4 | Decreased: 3/4 | Decreased: 2/2 |
| | | Absent: 0/4 | Absent: 0/4 | Absent: 0/4 | Absent: 0/2 |
| | Day 3 | Normal: 1/4 | Normal: 2/4 | Normal: 1/4 | Normal: 0/2 |
| | | Decreased: 3/4 | Decreased: 2/4 | Decreased: 3/4 | Decreased: 2/2 |
| | | Absent: 0/4 | Absent: 0/4 | Absent: 0/4 | Absent: 0/2 |
| Withdrawal | Day 1, | Normal: 4/4 | Normal: 4/4 | Normal: 4/4 | Normal: 2/2 |
| reflex | 2, and 3 | | | | |
| Proprioception | Day 1 | Normal: 1/4 | Normal: 1/3 | Normal: 2/4 | Normal: 1/2 |
| | | Decreased: 3/4 | Decreased: 2/3 | Decreased: 2/4 | Decreased: 1/2 |
| | | Absent: 0/4 | Absent: 0/3 | Absent: 0/4 | Absent: 0/2 |
| | Day 2 | Normal: 4/4 | Normal: 4/4 | Normal: 4/4 | Normal: 2/2 |
| | | Decreased: 0/4 | Decreased: 0/4 | Decreased: 0/4 | Decreased: 0/2 |
| | | Absent: 0/4 | Absent: 0/4 | Absent: 0/4 | Absent: 0/2 |
| | Day 3 | Normal: 4/4 | Normal: 4/4 | Normal: 4/4 | Normal: 2 |
| | | Decreased: 0/4 | Decreased: 0/4 | Decreased: 0/4 | Decreased: 0/2 |
| | | Absent: 0/4 | Absent: 0/4 | Absent: 0/4 | Absent: 0/2 |

All VAS and GCPS scores were below the cutoff of 4 cm and 6, respectively, for all subjects and timepoints. The injection site appeared normal in all animals. A subject in group 1 showed minimal reaction upon palpation at day 1 and a small red area at day 3 and they both resolved within 24 hours. A subject in group 3 had a small non-painful red area at day 14. All dogs, regardless of group, presented redness and swollen areas around the surgical incision that resolved within 2-3 days after surgery.

The results of the algometer are presented in Table 8. Mean plus or minus the standard deviation (±SD) force in Newton was recorded at the pressure applied on the surgical incision when the subject moved the leg from the algometer. The left side was the treated stifle.

TABLE 8
Algometer Measurements

| | Group | | | | | | | |
|---|---|---|---|---|---|---|---|---|
| | 1. High (25 μg) | | 2. Low (6.25 μg) | | 3. Local (50 μg) | | 4. Placebo | |
| Timepoint | L | R | L | R | L | R | L | R |
| Baseline | 7.4 ± 3.1 | 7.1 ± 3.7 | 6.2 ± 2.3 | 7.0 ± 2.3 | 5.7 ± 2.5 | 8.5 ± 2.4 | 8.7 ± 2.5 | 8.7 ± 1.5 |
| Day 1 | 7.4 ± 5.9 | 6.1 ± 2.3 | 5.0 ± 2.1 | 3.8 ± 1.9 | 9.2 ± 2.1 | 9.6 ± 6.3 | 3.7 ± 0.6 | 4.3 ± 1.1 |
| Day 2 | 5.4 ± 3.3 | 4.9 ± 2.1 | 5.0 ± 2.7 | 3.7 ± 2.0 | 8.5 ± 1.5 | 6.5 ± 3.5 | 7.7 ± 3.5 | 5.7 ± 1.3 |
| Day 3 | 5.3 ± 1.5 | 7.1 ± 1.8 | 5.9 ± 4.2 | 6.3 ± 1.5 | 7.7 ± 2.0 | 6.5 ± 3.4 | 8.5 ± 2.1 | 7.4 ± 1.4 |
| Day 14 | 5.3 ± 3.9 | 5.0 ± 2.3 | 2.9 ± 1.2 | 5.2 ± 1.5 | 6.6 ± 4.2 | 4.7 ± 3.5 | 3.7 ± 0.5 | 6.1 ± 1.1 |
| Day 28 | 5.8 ± 2.4 | 4.6 ± 3.5 | 4.5 ± 3.9 | 2.7 ± 2.1 | 4.4 ± 2.6 | 5.0 ± 1.9 | 3.4 ± 0.2 | 4.3 ± 0.1 |
| Day 56 | 7.0 ± 3.1 | 6.3 ± 1.2 | 4.9 ± 1.8 | 5.7 ± 1.7 | 10.9 ± 8.7 | 9.0 ± 4.6 | 7.0 ± 2.4 | 5.2 ± 0.8 |

The mean±SD force applied to the treated leg on day 1, 2, and 3 was similar to baseline values, except for the placebo group at day 1, which showed a trend of early reaction when a lower pressure was applied to the surgical site. In some cases, it was noticed that the animals reacted sooner than expected, especially after the first 2 weeks of the study. This behavior showed regardless of treatment and treated versus non-treated leg (i.e., group 2 day 14 and 28 on L and R, respectively; group 4 day 14 and 28 on L). This could have been due to the animal anticipating the stimulus or actual pain.

### Radiographic, CT, and MRI Findings

Orthogonal plane radiographs of the stifle were obtained for all dogs at all timepoints. After euthanasia, CT was obtained in 11 of the 14 dogs and MRI in all dogs. Metal artifact due to the presence of a broken screw prevented the evaluation of one MR study. Soft tissues could not be reliably assessed due to disruption secondary to removal of the plate. There was no evidence of implant failure or suboptimal osteotomy repair in any dog. Upon review of all imaging modalities, three patterns of healing emerged (Table 9a).

The delay of bone healing refers to a slower bone healing process as compared to the findings in the rest of the dogs in this study. This is not to be considered as an absolute delayed bone healing since the dogs were only enrolled in the study for 56 days

TABLE 9a
Summary of radiographic, CT, and MRI findings

| Pattern of healing | Dog ID | Group |
|---|---|---|
| No findings (N) | 2561 | 2. Low (6.25 μg) |
| | 2968 | 1. High (25 μg) |
| | 3007 | 3. Local (50 μg) |
| | 4681 | 2. Low (6.25 μg) |
| Soft tissue swelling with normal | 0271 | 1. High (25 μg) |
| bone healing (S) | 2272 | 3. Local (50 μg) |
| | 3837 | 1. High (25 μg) |
| | 5100 | 4. Placebo |
| | 5686 | 2. Low (6.25 μg) |
| Soft tissue swelling and delayed* | 3722 | 3. Local (50 μg) |
| bone healing (S&B) | 3743 | 2. Low (6.25 μg) |
| | 5635 | 1. High (25 μg) |
| | 7812 | 4. Placebo |
| | 9501 | 3. Local (50 μg) |

### No Findings (N) (4 Dogs)

At time of first recheck, mild fuzzy periosteal reaction was transiently observed, which by the time of the second recheck had mostly bridged with callus. In MRI, fluid intensity was absent from the osteotomy except laterally where a thin line persisted, and mild, diffuse marrow edema was present. A mild amount of excess joint fluid was considered within normal range.

Mild joint effusion and soft tissue swelling, as well as periosteal reaction was present at 27 days in dog 2968 (N). Osteotomy gap had bridged at 54 days. In CT, heterogeneous sclerosis was present adjacent to the osteotomy line, which has filled in, and in MR mild marrow edema remained in this region.

Soft Tissue Swelling with Normal Bone Healing (S) (5 Dogs)

For these dogs, soft tissue swelling in the region of the stifle+/−within the joint seemed accentuated, and persisted or worsened by the time of the second recheck. Bone healing was similar to the dogs classified as N, with bridging of the medial >lateral aspect of the osteotomy by 55 days.

Moderate joint effusion and soft tissue swelling were present at 28 days and progressed by 56 days in dog 5100 (S). However bone healing was normal, based on CT and MR. There was no evidence of septic arthritis or erosion of the subchondral bone in any of these dogs.

### Soft Tissue Swelling and Delayed Bone Healing (S&B) (5 Dogs).

Radiographic observations in this group included: Widening of the osteotomy line/bone resorption at 27 days or 55 days (5/5), fuzzy periosteal reaction at 27 days (2/5), best seen at caudal aspect of osteotomy line and laterally. In CT, cystic changes were seen along the osteotomy, more extensive medullary sclerosis (5/5). In MRI, these changes were not as pronounced as expected from CT images. Strong metal artifact was present in one of the five dogs (1/5) and prevented any interpretation of the MRI, and to a lesser degree, of the CT images. Cystic changes were only observed in the S&B group and were associated in every case with mild to moderate widening of the osteotomy gap from the time of surgery.

Radiographs immediately following surgery for dog 5635(S&B) were unremarkable. At 27 days, there was moderate soft tissue swelling in and around the joint. Fuzzy periosteal reaction was present near the caudal aspect of the osteotomy. At day 56 joint effusion and swelling had very mildly regressed, the osteotomy line was progressively wider, and the callus was smooth and organized. In CT, the osteotomy had not bridged with small (“cystic”) defects along that zone. In MRI, the osteotomy was filled with fluid-rich material (fibrocartilage vs. fluid), and intra- and extracapsular effusion was confirmed.

Porous tibial cortex was seen in two dogs (3743 and 9501), both of which exhibited the S&B pattern. Transverse CT image of the tibia distal to the osteotomy of dog 9501, in areas associated with abundant callus/periosteal reaction, the cortex was thin and appeared perforated by thin openings. This observation might be attributed to the healing process.

Table 9b shows a summary of the pattern of healing observed by group.

TABLE 9b
Pattern of Healing by Group

| | Pattern of healing | | |
|---|---|---|---|
| | No. of Dogs | | |
| Group | N | S | S&B |
| 1 (high dose) | 1 | 2 | 1 |
| 2 (low dose) | 2 | 1 | 1 |
| 3 (local) | 1 | 1 | 2 |
| 4 (control) | 0 | 1 | 1 |

### Necropsy Findings and Histopathology

During the removal of the TPLO plate, all stifles showed a normal callus and the proximal segment of the tibia was stable and fused to the distal portion. These findings were confirmed during necropsy.

No microscopic changes in the left stifle joints suggestive of RTX-related toxicity were identified. The incidences and severities of post-TPLO surgery changes in the left stifle joints showed no notable trends between left stifle joint groups. Any microscopic differences were interpreted to be related to the surgery or plane-of-section differences, and were considered unrelated to RTX.

Synovial hypertrophy/hyperplasia was noted in all left stifle joints examined. It was characterized by plump, variably piled synoviocytes with slightly more prominent fibrillary projections into the synovial spaces as compared to the control right stifle joints. Some control un-operated right stifle joints also occasionally had minimal synovial hypertrophy/hyperplasia typified by slightly plump, variably piled synoviocytes, but lacked increased fibrillary projections. The incidences and average severity grades of synovial hypertrophy/hyperplasia in left stifle joints showed no notable trends to suggest toxicity in left joint groups.

Soft tissue mononuclear inflammation was seen as small collections of lymphocytes, macrophages, and/or plasma cells or increased numbers of diffusely scattered mononuclear cells. The inflammation was commonly seen in the synovial lining subjacent to the synoviocytes, or in soft tissues that remained attached to the bone/joint. The incidences and average severity grades were not remarkably different between left joint groups.

Bone remodeling with increased osteoblasts/osteoclasts was characterized by irregular areas of bone/callous formation. Some areas of bone remodeling had increased cellularity due to increased numbers of osteoblasts and osteoclasts that outlined bone. The incidences and average severity grades showed no notable trends between left joint groups.

Clusters of irregular non-epiphyseal cartilage were noted in the left tibias, but were not typically seen in the non-operated right tibias. Presence of cartilage on the operated sides was presumptively interpreted to represent areas of residual cartilage in the physis which had been shifted/transposed by the TPLO surgery, and/or new cartilage formation. The amount of cartilage in each section was subjectively graded using the H&E and toluidine blue stains. The incidences and average severity grades of irregular non-epiphyseal cartilage in the left tibias were not notably different between left joint groups. Any differences could also be influenced by differences in physeal cartilage amounts/extent of physeal closure between dogs, and by plane-of-section variabilities.

A collagen score for each of the stifle joint slides stained with picosirius red was assigned based on the qualitative estimate of collagen present on the slide. The average of the three slides per joint was recorded. An overall collagen score for the left and right stifle joints for each group was calculated by averaging the individual collagen scores. The collagen scores for the left stifle joints were not notably different between groups. The average collagen scores of groups 1-4 for the operated left legs were slightly higher than the corresponding un-operated right leg group average collagen scores. Higher collagen in the operated legs seemed consistent with post-surgical healing processes.

TABLE 10
Picosirius red collagen scores

| | Group | | | |
|---|---|---|---|---|
| | 1. High | 2. Low | 3. Local | |
| | (25 μg) | (6.25 μg) | (50 μg) | 4. Placebo |
| Average collagen | 2.3 | 2.3 | 2.4 | 2.2 |
| score right leg | | | | |
| Average collagen | 2.9 | 2.6 | 3.0 | 3.0 |
| score left leg | | | | |

Other sporadic changes in the left stifle joints of some dogs included soft tissue bone fragments, soft tissue hemorrhage, skeletal muscle degeneration, fibrocartilage degeneration, periosteal disruption, periosteal thickening/fibrosis, and bone marrow fibrosis/fibroplasia. There were no remarkable incidence or severity patterns to suggest that these changes were indicative of potential RTX toxicity.

TABLE 11
Select microscopic findings - left stifle joint

| | Group | | | |
|---|---|---|---|---|
| | 1. High | 2. Low | 3. Local | |
| | (25 μg) | (6.25 μg) | (50 μg) | 4. Placebo |
| No. Animals Examined | 4 | 4 | 4 | 2 |
| Left stifle joint (No. | 4 | 4 | 4 | 2 |
| Examined) | | | | |
| Screw holes with intraluminal | (4) | (4)^{a} | (4) | (2) |
| hemorrhage | | | | |
| Mild | 4 | 4 | 4 | 2 |
| Synovial | (4) | (4)^{a} | (4) | (2) |
| hypertrophy/hyperplasia | | | | |
| Minimal | 0 | 0 | 0 | 0 |
| Mild | 4 | 3 | 3 | 2 |
| Moderate | 0 | 1 | 1 | 0 |
| Soft tissue mononuclear | (4) | (4)^{a} | (4) | (2) |
| inflammation | | | | |
| Minimal | 3 | 0 | 2 | 0 |
| Mild | 1 | 4 | 2 | 2 |
| Bone remodeling with | (4) | (4)^{a} | (4) | (2) |
| increased osteoblasts/ | | | | |
| osteoclasts | | | | |
| Minimal | 2 | 1 | 0 | 0 |
| Mild | 2 | 2 | 2 | 2 |
| Moderate | 0 | 1 | 2 | 0 |
| Non-epiphyseal cartilage | (4) | (4)^{a} | (4) | (2) |
| Minimal | 1 | 1 | 0 | 0 |
| Mild | 1 | 1 | 2 | 2 |
| Moderate | 2 | 2 | 2 | 0 |
| Collagen (picosirius red stain) | (4) | (4)^{a} | (4) | (2) |
| Average collagen score | 2.9 | 2.6 | 3.0 | 3.0 |

^{a}number in parentheses indicates the number of dogs in the group with the finding

### Quantification of CGRP and SP Immunopositive Nerve Fibers

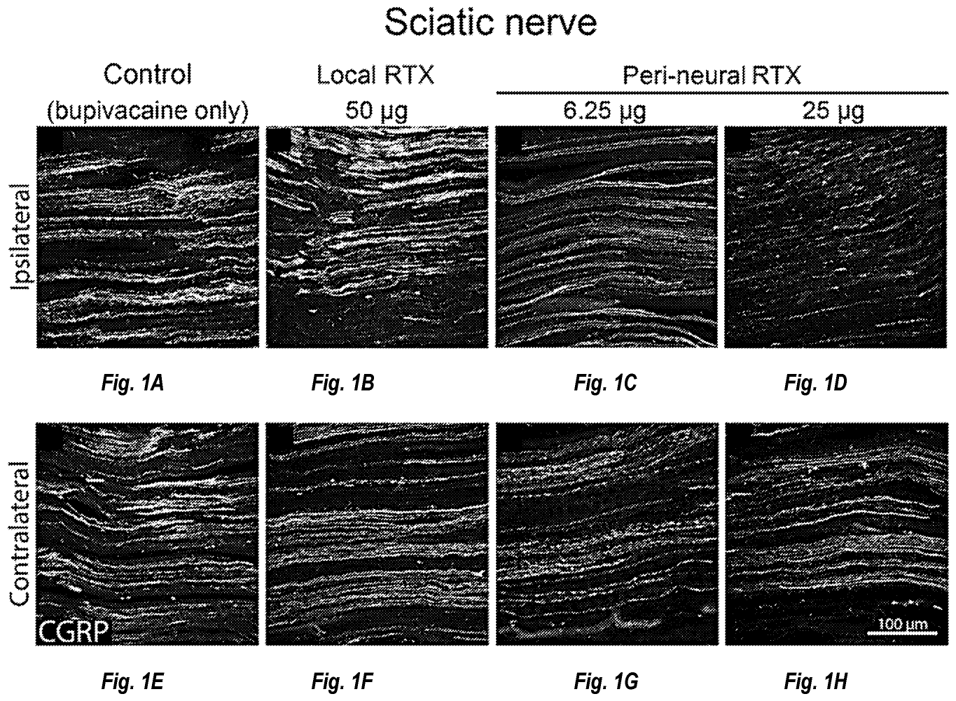

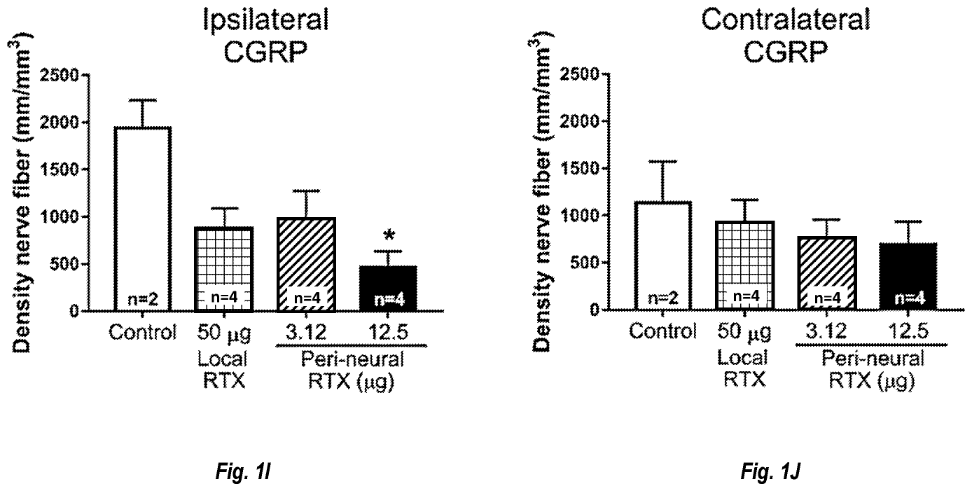

The effects of different routes of administration and/or different doses of RTX on the density of CGRP immunopositive axons in the ipsilateral (left) and contralateral (right) sciatic nerves are shown in FIG. 1. Representative confocal images revealed a robust presence of CGRP, which may be visualized as single long or bundles of axons placed horizontally on the focal plane. Confocal images show that tibial plateau leveling osteotomy (TPLO) surgery by itself tended to increase the density of CGRP nerve axons (FIG. 1A) compared to the contralateral nerve (FIG. 1E); however, this effect was not statistically different (p=0.2681). RTX treatment resulted in a reduction of CGRP immunopositive axons in the ipsilateral sciatic nerve as compared to control treatment. Quantitative analysis revealed that both local and peri-neural administration with RTX reduced the density of CGRP nerve axons (FIG. 1I) in the ipsilateral sciatic nerve as compared to control treatment. However, statistical significance was observed only with the dose of 12.5 μg (25 μg total; p=0.0184) per nerve of RTX. FIGS. 1E-H show the effect of different treatment and doses of RTX on the density of CGRP nerve axons innervating the contralateral (right) sciatic nerve from animals post-TPLO. However, as expected, no differences in the density of CGRP nerve axons innervating the contralateral sciatic nerve were observed in the RTX treated group as compared to control treatment (FIG. 1J) (p=0.9565, p=0.7911, p=0.7229, respectively).

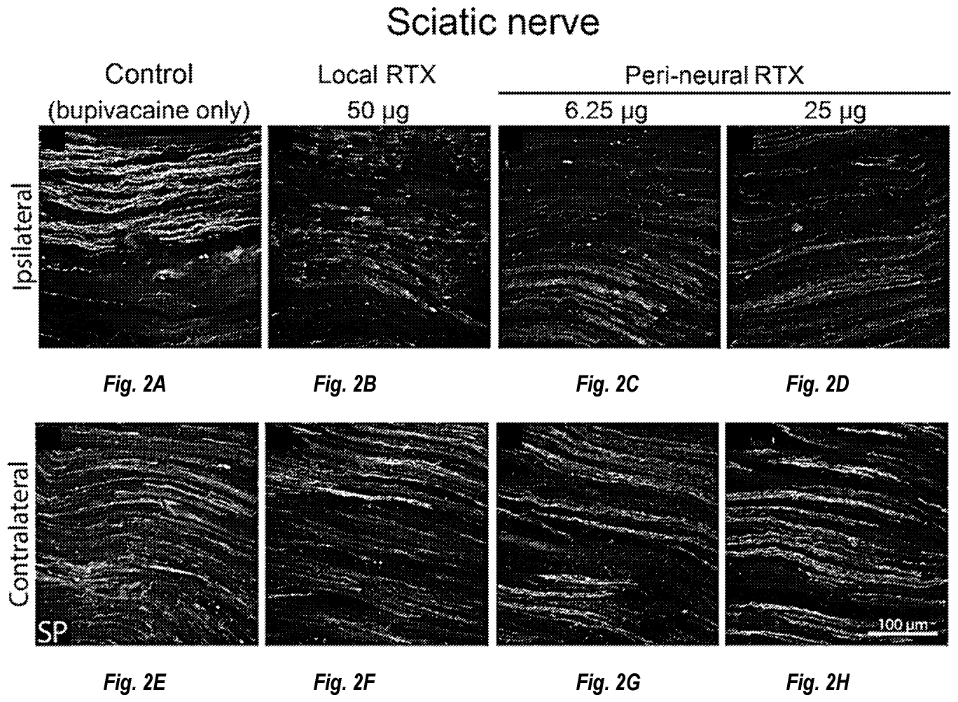

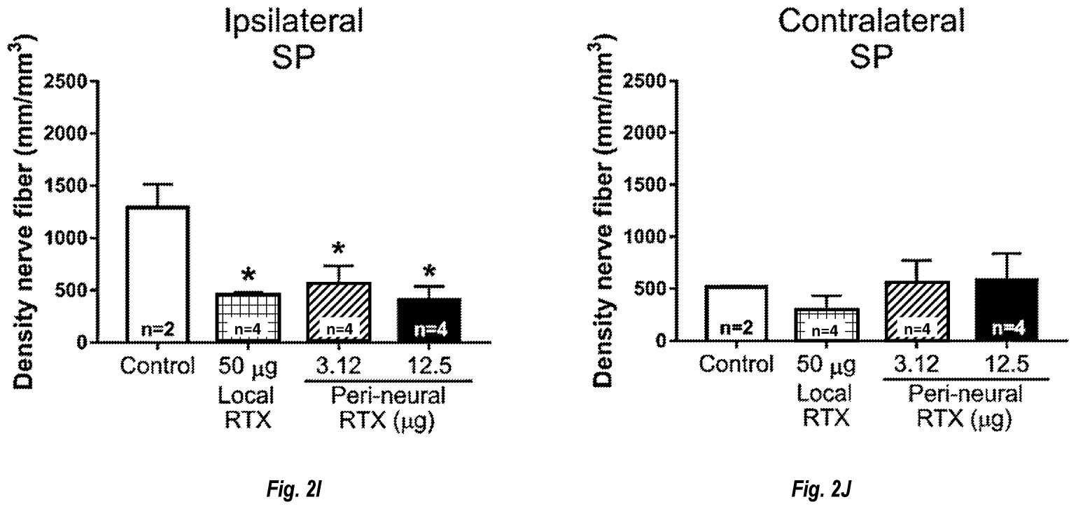

FIG. 2 shows the effect of different treatment and doses of RTX on the density of SP nerve axons innervating the ipsilateral and contralateral sciatic nerves. Representative confocal images show that TPLO surgery by itself increased the density of SP nerve axons (FIG. 2A) compared to the contralateral nerve (FIG. 2E); however, this effect was not statistically different (p=0.0737). RTX treatments resulted in a drastic reduction in the density of SP axons in the ipsilateral sciatic nerve (FIG. 2A-D). As expected, no effect of RTX was observed in the contralateral side (FIG. 2E-H) as compared to control treatment. Quantitative analysis revealed that local administration (p=0.0155) and peri-neural treatment at doses of 3.12 (6.25 μg total; p=0.0335) and 12.5 μg (25 μg total; p=0.0112) per nerve of RTX produced a statistically significant reduction on the density of SP axons of the ipsilateral sciatic nerve as compared to control treatment (FIG. 2I). As expected, treatment with RTX did not affect the density of SP nerve axons in the contralateral sciatic nerve as compared to control treatment (FIG. 11J) (p=0.9122, p=0.9995, p=0.9971, respectively).

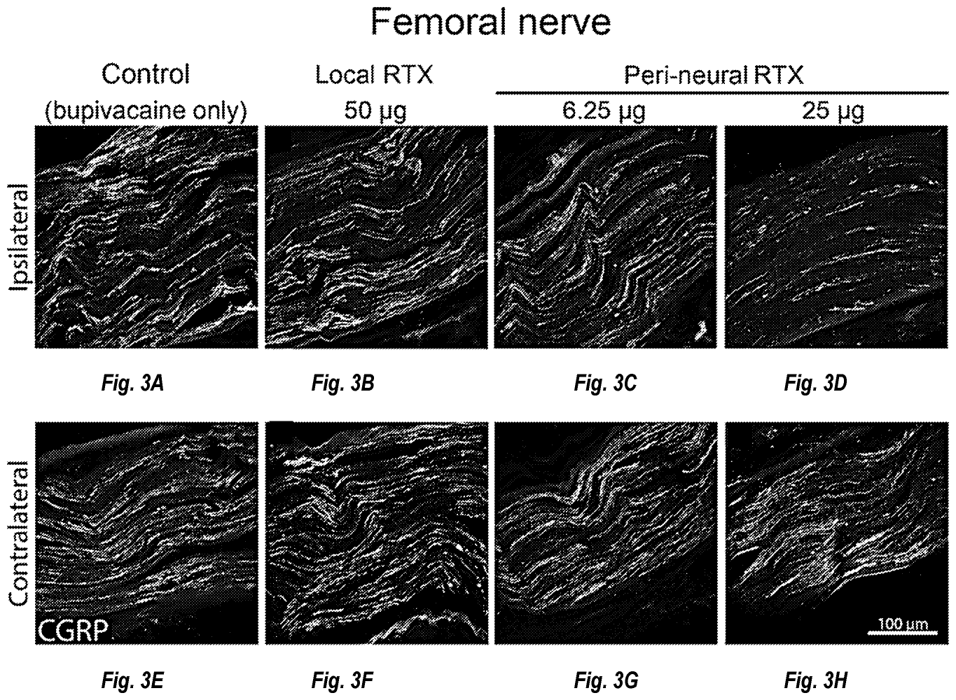

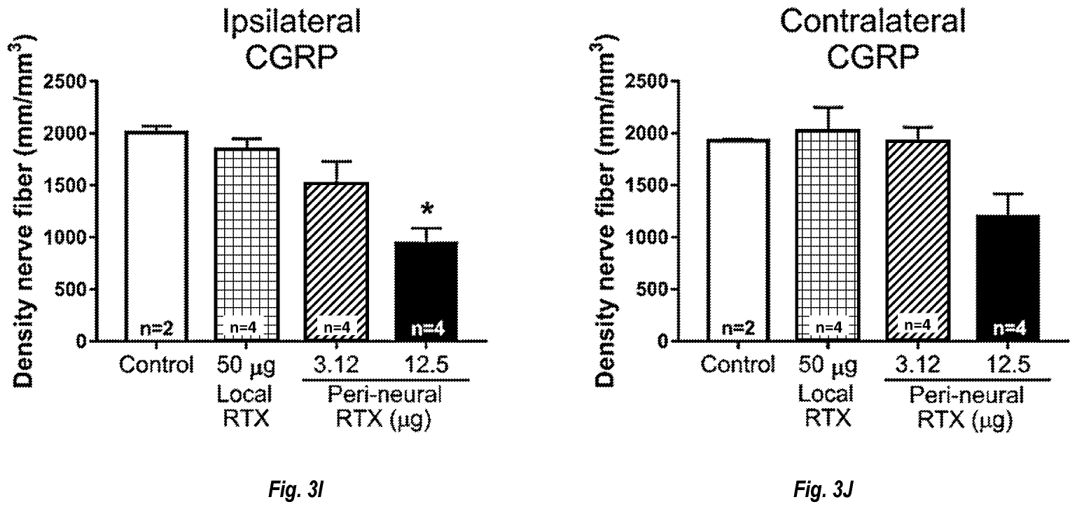

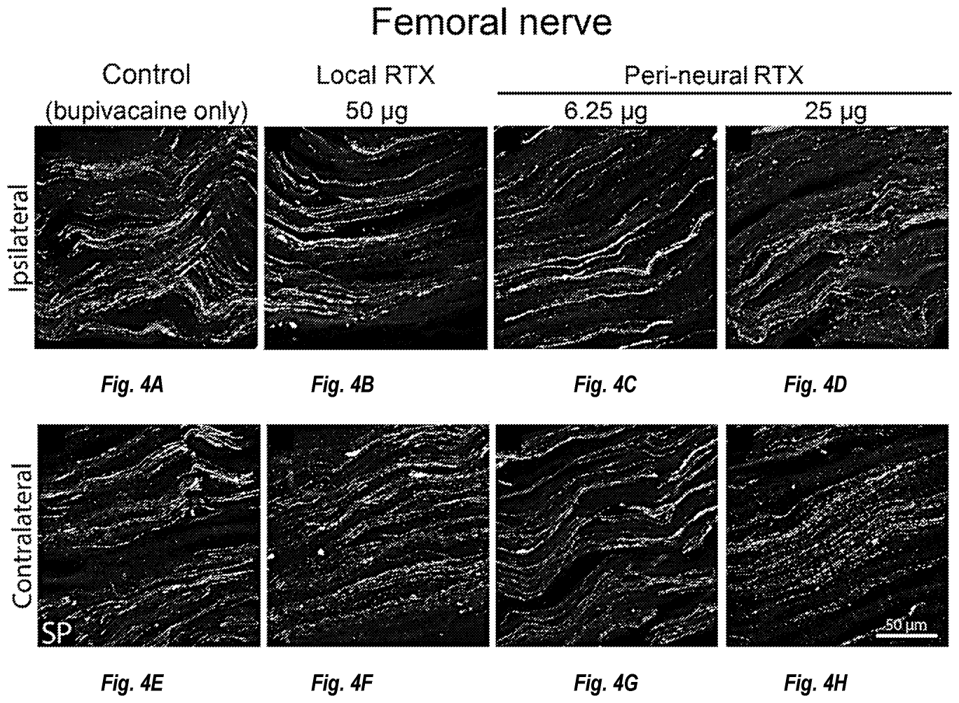

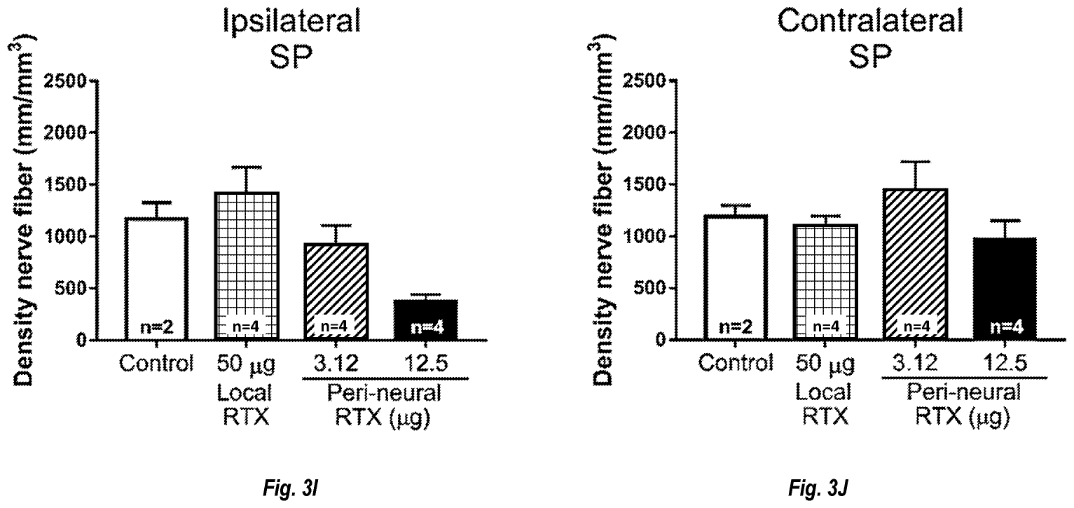

FIG. 3 shows the effect of either local or pen-neural treatment with RTX on the density of CGRP nerve axons innervating the ipsilateral (FIG. 3A-D) and the contralateral (FIG. 3E-H) femoral nerves from dogs with TPLO. Confocal and quantitative analysis revealed TPLO surgery by itself did not increase the density of CGRP nerve axons in the femoral nerve (FIG. 3A) compared to the contralateral femoral nerve (FIG. 3E) (p=0.3391). Peri-neural treatment with RTX at 12.5 μg (25 μg total; p=0.0125) per nerve statistically reduced the density of CGRP nerve axons in the ipsilateral femoral nerve (FIG. 12I). In contrast, any treatment of RTX treatment did not statistically reduce the density of CGRP nerve axons in the contralateral femoral nerve as compared to control treatment (FIG. 3J) (p=0.9914, p>0.9999, p=0.1760, respectively).

FIG. 4 illustrates the effect of RTX on SP density in nerve fibers innervating the ipsilateral (FIG. 4A-D) and contralateral (FIG. 4E-H) femoral nerves from dogs post-TPLO. TPLO surgery by itself did not increase the density of SP nerve axons in the femoral nerve in the operated leg (FIG. 4A) compared to density of these axons in the contralateral femoral nerve (FIG. 4E) (p=0.9151). Quantitative analysis revealed that pen-neural RTX at both doses (3.12 μg and 12.5 μg per nerve) resulted in a marked trend in reducing density of SP nerve axons; however, this reduction was not statistically significant (p=0.8519, p=0.1216, respectively) (FIG. 4I). As expected, RTX treatment did not have an effect on the density of SP nerve axons in the contralateral femoral nerve (p=0.9907, p=0.8812, p=0.9066, respectively) (FIG. 4J).

### SUMMARY OF RESULTS

All RTX treatments (femoral/sciatic nerve block at 3.12 μg per nerve (6.25 μg RTX total) and 12.5 μg per nerve (25 μg RTX total), and local application of a gauze loaded with 50 μg) post-osteotomy were well tolerated. One dog, which was in estrus at the time of the treatment, developed hypotension, transient cardiac arrythmias and decreased in end-tidal CO₂. All dogs recovered uneventfully. A limitation of this study design was the inability to quantify the exact amount of RTX to which the dogs in group 3 (local) were exposed. Although the gauze used during the application was saturated with 50 ug of RTX and it was left undisturbed for 10 min, it is likely that the osteotomy site was exposed to a lower total dose.

The perineural injection of RTX at 3.12 μg RTX per nerve (6.25 μg RTX total) and 12.5 μg RTX per nerve (25 μg RTX total) in dogs undergoing TPLO surgery shown short-term efficacy in treating post-operative pain.

Anesthetic management was similar among all dogs in the 4 groups. Additional interventions (intraoperative propofol and hydromorphone) due to a higher sympathetic stimulation and/or light plane of anesthesia were recorded for dogs that received RTX. However, these dogs received less dobutamine, used to treat hypotension, especially after RTX was administered.

Within 2 hours of surgery, all dogs in group 1 used the operated leg (two dogs normally and the other two intermittently) and three out of four dogs in group 2 used it intermittently. In contrast, no dogs in groups 3 or 4 used the operated leg until the following day.

Post-operatively, all dogs in groups 1 and 2 showed polypnea, which persisted until the body temperature decreased to 94-95° F. In some dogs this decrease in body temperature was present up to 3 days after the treatment; however, clinically all these dogs acted normally (normal food/water consumption and physical activity) and their blood work was within normal limits.

Regardless of dose, plasma concentrations of RTX did not reach detectable levels until at least 90 minutes after administration, except for one dog in group 1. These plasma concentrations were generally associated with an increase in heart rate and blood pressure. In group 2, RTX plasma levels fell to below detectable after 3-4 hours, whereas for group 1 levels were still detectable at 8 hours. Plasma levels were below the quantifiable limit at all timepoints for all dogs in groups 3 and 4.

Three healing patterns were noted on radiograph, CT, and MRI examinations: No findings (N), Soft tissue swelling with normal bone healing (S), and Soft tissue swelling and delayed bone healing (S&B). The delay of bone healing refers to a slower bone healing process when compared to the findings in the rest of the dogs in this study. The distribution of these patterns among the treatment groups suggested that RTX did not adversely affect the bone healing process. Moreover, none of the radiographic findings were associated with clinical signs or adverse effects.

Histopathologic examination revealed synovial hypertrophy/hyperplasia, soft tissue mononuclear inflammation, bone remodeling with increased number of osteoblasts/osteoclasts, and presence of new collagen in all left stifle joints. These changes were related to the surgery or plane-of-section differences, and were considered unrelated to RTX. No microscopic changes suggestive of RTX-related toxicity were identified.

TPLO surgery resulted in a trend of increased density of CGRP and SP nerve axons in the sciatic nerve, but not in the femoral nerve. CGRP significantly decreased after peri-neural RTX injection of 12.5 μg per nerve (25 μg total) around the sciatic and femoral nerves. SP decreased significantly in the sciatic nerve after peri-neural RTX injection of 12.5 μg per nerve (25 μg total), 3.12 μg per nerve (6.25 μg total), and local application of a gauze loaded with 50 μg of RTX. SP also decreased in both groups receiving the peri-neural RTX injection (group 1 and 2), but this decrease was not significant.

What is claimed is:

1. A method of preparing a subject for surgery, comprising perineurally administering RTX to a subject in need of surgery.

2. Resiniferatoxin (RTX) for use in a method of preparing a subject for surgery, the method comprising perineurally administering RTX to a subject in need of surgery.

3. The method or RTX for use of claim 1 or 2, wherein the method provides a peri-surgical or a post-surgical benefit to the subject.

4. The method or RTX for use of any one of the preceding claims, wherein the method reduces post-surgical pain in the subject.

5. The method or RTX for use of any one of the preceding claims, wherein the method accelerates post-surgical recovery time of the subject.

6. The method or RTX for use of any one of the preceding claims, wherein the method reduces the amount of peri-surgical anesthetic administered to the subject.

7. The method or RTX for use of any one of the preceding claims, wherein the method reduces the amount of post-surgical anesthetic administered to the subject.

8. The method or RTX for use of any one of the preceding claims, wherein the method reduces inflammation caused by trauma at a surgical site.

9. The method or RTX for use of any one of the preceding claims, wherein the method reduces cytokine release caused by trauma at a surgical site.

10. The method or RTX for use of any one of the preceding claims, wherein nerve signaling pathways are ablated in the subject.

11. The method or RTX for use of any one of the preceding claims, wherein the subject is being prepared for a surgical procedure.

12. The method or RTX for use of any one of the preceding claims, wherein the subject is placed under anesthesia prior to the administration of RTX.

13. The method or RTX for use of any one of the preceding claims, wherein the surgery occurs within 1, 2, 3, 4, 5, or 6 hours of administering RTX.

14. The method or RTX for use of any one of the preceding claims, wherein the subject is in need of an orthopedic surgery or an amputation.

15. The method or RTX for use of any one of the preceding claims, wherein the subject is in need of surgical intervention on a bone in the back, hand, arm or leg.

16. The method or RTX for use of any one of the preceding claims, wherein the RTX is administered as an infiltration.

17. The method or RTX for use of any one of the preceding claims, wherein the RTX is administered to a single site.

18. The method or RTX for use of any one of the preceding claims, wherein the RTX is administered to a plurality of sites.

19. The method or RTX for use of any one of the preceding claims, wherein the RTX is administered locally to a surgical site.

20. The method or RTX for use of any one of the preceding claims, wherein RTX is administered as a nerve block.

21. The method or RTX for use of any one of the preceding claims, wherein RTX is administered as a nerve block at the sciatic nerve and/or the femoral nerve.

22. The method or RTX for use of any one of the preceding claims, wherein the subject is a mammal.

23. The method or RTX for use of any one of the preceding claims, wherein the subject is a cat, dog, horse, pig, ruminant, cow, sheep, goat, or domesticated mammal.

24. The method or RTX for use of any one of the preceding claims, wherein the subject is a human.

25. The method or RTX for use of any one of the preceding claims, wherein the method comprises administering a dose of 0.1 μg to 100 μg of RTX, or a dose of 2 μg to 50 μg of RTX.

26. The method or RTX for use of claim 25 wherein the dose of RTX ranges from 0.1-1 μg, 1-2 μg, 2-5 μg, 5-10 μg, 10-20 μg, 20-30 μg, 30-40 μg, 40-50 μg, 50-60 μg, 60-70 μg, 70-80 μg, 80-90 μg, or 90-100 μg.

27. The method or RTX for use of any one of the preceding claims, wherein the method comprises administering a pharmaceutical formulation comprising the RTX and a pharmaceutically acceptable carrier.

28. The method or RTX for use of claim 27, wherein the pharmaceutically acceptable carrier comprises water.

29. The method or RTX for use of claim 27 or 28, wherein the pharmaceutically acceptable carrier comprises polysorbate 80.

30. The method or RTX for use of any one of claims 27-29, wherein the pharmaceutically acceptable carrier comprises polyethylene glycol.

31. The method or RTX for use of any one of claims 27-30, wherein the pharmaceutically acceptable carrier comprises a sugar or sugar alcohol.

32. The method or RTX for use of claim 27-31, wherein the pharmaceutically acceptable carrier comprises mannitol.

33. The method or RTX for use of claim 27-32, wherein the pharmaceutically acceptable carrier comprises dextrose.

34. The method or RTX for use of any one of claims 27-33, wherein the pharmaceutically acceptable carrier comprises a pharmaceutically acceptable buffer.

35. The method or RTX for use of claim 27-34, wherein the pharmaceutically acceptable carrier comprises a phosphate buffer.

36. The method or RTX for use of any one of claims 27-35, wherein the pharmaceutical formulation has a pH in the range of 6 to 7.6.

37. The method or RTX for use of claim 36, wherein the pharmaceutical formulation has a pH in the range of 6 to 6.4, 6.3 to 6.7, 6.4 to 6.8, 6.8 to 7.2, 7 to 7.4, or 7.2 to 7.6.

38. The method or RTX for use of claim 36, wherein the pharmaceutical formulation has a pH of 6.5 or 7.2.

39. The method or RTX for use of any one of claims 27-38, wherein the pharmaceutically acceptable carrier comprises a pharmaceutically acceptable salt.

40. The method or RTX for use of claim 39, wherein the pharmaceutically acceptable salt is NaCl.

41. The method or RTX for use of any one of claims 27-40, wherein the concentration of RTX in the pharmaceutical formulation is in the range of 0.02 to 300 μg/ml.

42. The method or RTX for use of claim 41, wherein the concentration of RTX in the pharmaceutical formulation is in the range of 0.02-0.1 μg/ml, 0.1-1 μg/ml, 1-5 μg/ml, 5-10 μg/ml, 10-20 μg/ml, 20-50 μg/ml, 50-100 μg/ml, 100-150 μg/ml, 150-200 μg/ml, 200-250 μg/ml, or 250-300 μg/ml.

43. The method or RTX for use of claim 41, wherein the concentration of RTX in the pharmaceutical formulation is in the range of 150 to 250 μg/ml, or is about 200 μg/ml.

44. The method or RTX for use of claim 41, wherein the concentration of RTX in the pharmaceutical formulation is in the range of 0.1-200 μg/ml, optionally wherein the concentration of RTX in the pharmaceutical formulation is in the range of 0.1-50 μg/ml.

45. The method or RTX for use of any one of the preceding claims, wherein the RTX is administered in an injection volume of 0.05-10 ml, optionally wherein the injection volume is in the range of 0.05-0.2 ml, 0.2-0.5 ml, 0.5-1 ml, 1-2 ml, 2-5 ml, or 5-10 ml.

46. The method or RTX for use of any one of the preceding claims, wherein the method further comprises administering a local anesthetic.

47. The method or RTX for use of claim 46, wherein the local anesthetic is an amino-amide anesthetic.

48. The method or RTX for use of claim 46, wherein the local anesthetic is bupivacaine.

49. The method or RTX for use of any one of the preceding claims, wherein the method is a method of reducing post-operative pain.

50. The method or RTX for use any one of the preceding claims, wherein the method is a method of accelerating post-surgical recovery time of the subject.
